SEQUENCE LISTING (25 sequences)

SEQ ID NO: 1 - protein, 117 aa, Artificial [sequence omitted]

SEQ ID NO: 2 - protein, 107 aa, Artificial [sequence omitted]

SEQ ID NO: 3 - protein, 8 aa, Artificial
  Synthetic
GFTFSNFG

SEQ ID NO: 4 - protein, 8 aa, Artificial
  Synthetic
ISGGGRDT

SEQ ID NO: 5 - protein, 10 aa, Artificial
  Synthetic
VKWGNIYFDY

SEQ ID NO: 6 - protein, 6 aa, Artificial
  Synthetic
LSINTF

SEQ ID NO: 7 - protein, 3 aa, Artificial
  Synthetic
AAS

SEQ ID NO: 8 - protein, 9 aa, Artificial
  Synthetic
QQSSNTPFT

SEQ ID NO: 9 - protein, 444 aa, Artificial
  Synthetic
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, Artificial
  Synthetic
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 127 aa, Artificial
  Synthetic
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSS

SEQ ID NO: 12 - protein, 108 aa, Artificial
  Synthetic
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKR

SEQ ID NO: 13 - protein, 123 aa, Artificial
  Synthetic
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVAS

SEQ ID NO: 14 - protein, 8 aa, Artificial
  Synthetic
GFTFNDYA

SEQ ID NO: 15 - protein, 8 aa, Artificial
  Synthetic
ISWNSDSI

SEQ ID NO: 16 - protein, 20 aa, Artificial
  Synthetic
AKDNHYGSGSYYYYQYGMDV

SEQ ID NO: 17 - protein, 6 aa, Artificial
  Synthetic
QSVSSN

SEQ ID NO: 18 - protein, 9 aa, Artificial
  Synthetic
GLYALASER

SEQ ID NO: 19 - protein, 9 aa, Artificial
  Synthetic
QHYINWPLT

SEQ ID NO: 20 - protein, 8 aa, Artificial
  Synthetic
GFTFDDYT

SEQ ID NO: 21 - protein, 8 aa, Artificial
  Synthetic
ISWNSGSI

SEQ ID NO: 22 - protein, 16 aa, Artificial
  Synthetic
AKDNSGYGHYYYGMDV

SEQ ID NO: 23 - protein, 453 aa, Artificial
  Synthetic
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 214 aa, Artificial
  Synthetic
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 449 aa, Artificial
  Synthetic
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

(19) United States
(12) Patent Application Publication
(10) Pub. No.: US 2023/0374160 A1
(43) Pub. Date: Nov. 23, 2023

(54) COMBINATION OF ANTIBODIES FOR TREATING CANCER WITH REDUCED CYTOKINE RELEASE SYNDROME

(71) Applicant: Regeneron Pharmaceuticals, Inc., Tarrytown, NY (US)

(72) Inventors: Aynur Hermann, New York, NY (US); Erica Ullman, Yorktown Heights, NY (US); Min Zhu, Ossining, NY (US); Masood Khaksar Toroghi, Congers, NY (US)

(21) Appl. No.: 18/247,226

(22) PCT Filed: Oct. 1, 2021
(86) PCT No.: PCT/US21/53065

§ 371 Date: Mar. 29, 2023

Related U.S. Application Data
(60) Provisional application No. 63/240,759, filed on Sep. 3, 2021.
(60) Provisional application No. 63/086,658, filed on Oct. 2, 2020.

(51) Int. Cl. C07K 16/46; C07K 16/28; A61P 35/00

(52) U.S. Cl. CPC C07K 16/468; C07K 16/2818; A61P 35/00; C07K 2317/31; C07K 2317/565

(57) ABSTRACT

This disclosure provides methods for treating cancer and for ameliorating cytokine release syndrome in a subject with a tumor. The methods include administering to a subject in need thereof a bispecific anti-CD20/anti-CD3 antibody before administering to the subject an anti-PD-1 antibody. The disclosed methods represent effective therapies for cancer, e.g, B-cell malignancies, with mitigation of the potential life-threatening effects of cytokine release syndrome (CRS).

### FIELD

The present disclosure relates to methods for treating cancer and methods for ameliorating cytokine release syndrome in a subject with a tumor or tumor cells. The methods include administering to a subject in need thereof a bispecific antibody that binds to CD20 and CD3 before administering to the subject an antibody that binds to PD-1.

### BACKGROUND

B-cell cancers are a group of heterogeneous cancers of the white blood cells known as B-lymphocytes and include leukemias (located in the blood) and lymphomas (located in the lymph nodes). B-cell lymphomas include, but are not limited to, non-Hodgkin's lymphoma (NHL) and Hodgkin's lymphoma (HL). Lymphomas are divided into indolent (slow-growing) or aggressive lymphomas. A common indolent lymphoma is follicular lymphoma, while the most common aggressive lymphoma is diffuse large B-cell lymphoma. B-cell leukemias include, but are not limited to, acute lymphoblastic leukemia, hairy cell leukemia, and B-cell chronic lymphocytic leukemia.

Most B-cell cancers express CD20 on the cell surface of mature B-cells. Methods for treating cancer by targeting CD20 are known in the art. For example, the chimeric anti-CD20 monoclonal antibody rituximab has been used or suggested for use in treating cancers such as NHL, chronic lymphocytic leukemia (CLL) and small lymphocytic lymphoma (SLL), either as monotherapy but more typically in combination with chemotherapy. Although anti-CD20 tumor-targeting strategies have shown great promise in clinical settings, not all patients respond to anti-CD20 therapy, and some patients have been shown to develop resistance to or exhibit incomplete responses to anti-CD20 therapy (e.g., partial depletion of peripheral B-cells), for reasons that are not well understood (but which typically do not include loss of CD20 expression). Some patients relapse with a more aggressive phenotype or chemotherapy-resistant disease. Many patients with aggressive lymphomas have poor prognosis and less than 50% chance of relapse-free survival. The prognosis for patients who relapse or are refractory to therapy remains dismal with median survival after salvage therapy of 2 to 8 months. In addition, high-dose chemotherapy leads to severe adverse side effects. Thus, there is a high unmet need for therapies that are effective in preventing relapse and have less side effects for patients with B-cell cancers.

Activation of T cells naturally occurs through their T cell receptors (TCR) in complex with CD3 subunits (CD3γε-CD3δε-CD3ζζ) by ligation to cognate peptides displayed on major-histocompatibility complex (MHC) molecules on antigen-presenting cells (APC). Antibodies against CD3 have been shown to cluster CD3 on T cells, thereby causing T cell activation in a manner similar to the engagement of the TCR by peptide-loaded MHC molecules. Bispecific monoclonal antibodies designed to target both CD20 and CD3 bridge CD20-expressing cells with cytotoxic T cells, resulting in CD20-directed polyclonal T cell killing.

Programmed death-1 (PD-1) receptor signaling in the tumor microenvironment plays a key role in allowing tumor cells to escape immune surveillance by the host immune system. Blockade of the PD-1 signaling pathway has demonstrated clinical activity in patients with multiple tumor types, and antibody therapeutics that block PD-1 (e.g., nivolumab and pembrolizumab) have been approved for the treatment of metastatic melanoma and metastatic squamous non-small cell lung cancer. Recent data has demonstrated the clinical activity of PD-1 blockade in patients with aggressive NHL and Hodgkin's lymphoma (Lesokhin, et al. 2014, Abstract 291, 56th ASH Annual Meeting and Exposition, San Francisco, Calif.; Ansell et al. 2015, N. Engl. J. Med. 372(4):311-9).

However, the management of the toxicities of cancer immunotherapy is a challenging clinical problem. Mitigating cytokine release syndrome (CRS) or infusion-related reaction (IRR) is a hallmark of administering certain treatment modalities, for example, chimeric antigen receptor (CAR) T cells and bispecific antibodies targeting T cells. T cell redirecting therapies, including CAR T cell therapy and CD20×CD3 bispecific antibodies, have been associated with increases in serum cytokines in patients with B-cell non-Hodgkin lymphoma, which may lead to a systemic inflammatory response, CRS (Shimabukuro-Vornhagen et al., J Immunother Cancer, 2018, 6(1):56). Low grade CRS is generally treated symptomatically with antihistamines, antipyretics, and fluids. Severe CRS can represent a life-threatening adverse event that requires prompt and aggressive treatment. Reduction of tumor burden, limitations on the dose of administered therapy, and premedication with steroids have reduced the incidence of severe CRS, as have the use of anti-cytokine treatments. However, the use of dose limitations and treatments to minimize cytokine activity can have detrimental effects on the efficacy of the immunotherapy. Additionally, targeting PD-1/PD-L1 has the potential to promote T cell activation and inhibit escape from PD-1 mediated tumor immune surveillance, but could also increase cytokine release, resulting in a higher incidence and severity of CRS.

Accordingly, there remains a strong need for effective therapies for cancer, e.g., B-cell malignancies, while mitigating the potential life-threatening effects of CRS without negatively impacting the therapeutic benefits of the therapy.

### SUMMARY

The disclosed technology addresses the need mentioned above in various aspects. In one aspect, this disclosure provides a method of treating or inhibiting the growth of a tumor, including: (a) selecting a subject with cancer; (b) administering to the subject a therapeutically effective amount of a bispecific anti-CD20/anti-CD3 antibody or antigen-binding fragment thereof including a first antigen-binding arm that specifically binds CD20 and a second antigen-binding arm that specifically binds CD3; and (c) after step (b), administering to the subject an antibody or antigen-binding fragment thereof that specifically binds programmed death 1 (PD-1); wherein the method treats or inhibits the growth of a tumor and ameliorates cytokine release syndrome (CRS) in the subject.

In another aspect, this disclosure provides a method of ameliorating cytokine release syndrome (CRS) in a subject with a tumor, including: (a) selecting a subject with cancer; (b) administering to the subject a therapeutically effective amount of a bispecific anti-CD20/anti-CD3 antibody or antigen-binding fragment thereof including a first antigen-binding arm that specifically binds CD20 and a second antigen-binding arm that specifically binds CD3; and (c) after step (b), administering to the subject an antibody or antigen-binding fragment thereof that specifically binds programmed death 1 (PD-1).

In some embodiments, the step of administering to the subject an anti-PD-1 antibody or antigen-binding fragment thereof further includes administering to the subject the anti-PD-1 antibody or antigen-binding fragment thereof in combination with the bispecific antibody or antigen-binding fragment thereof. In some embodiments, the bispecific antibody or antigen-binding fragment thereof is administered to the subject at least about 1 week prior to administering the anti-PD-1 antibody or antigen-binding fragment thereof. In some embodiments, each of the bispecific antibody or antigen-binding fragment thereof and the anti-PD-1 antibody or antigen-binding fragment thereof is administered in one or more doses to the subject.

In some embodiments, the first dose of the bispecific antibody or antigen-binding fragment thereof is administered to the subject about 1 week, about 2 weeks, about 3 weeks, about 4 weeks, about 5 weeks, about 6 weeks, about 7 weeks, or about 8 weeks prior to administering the first dose of the anti-PD-1 antibody or antigen-binding fragment thereof. In some embodiments, the first dose of the bispecific antibody or antigen-binding fragment thereof is administered to the subject about 5 weeks prior to administering the first dose of the anti-PD-1 antibody or antigen-binding fragment thereof. In some embodiments, the bispecific antibody or antigen-binding fragment thereof is administered to the subject in one or more doses of about 0.1 mg/kg to about 15 mg/kg of body weight of the subject. In some embodiments, the bispecific antibody or antigen-binding fragment thereof is administered to the subject in one or more doses of about 1 mg to about 800 mg. In some embodiments, the bispecific antibody or antigen-binding fragment thereof is administered to the subject once a day, once every two days, once every three days, once every five days, once every week, once every two weeks, once every three weeks, or once every four weeks. In some embodiments, each dose of the one or more doses of the bispecific antibody or antigen-binding fragment thereof is administered 0.5 to 12 weeks after the immediately preceding dose.

In some embodiments, at least one of the one or more doses of the bispecific antibody or antigen-binding fragment thereof includes a dose having a greater amount of the bispecific antibody or antigen-binding fragment thereof than the immediately preceding dose thereof. In some embodiments, at least one of the one or more doses of the bispecific antibody or antigen-binding fragment thereof is administered in two or more split doses. In some embodiments, at least one of the two or more split doses includes the identical amount of the bispecific antibody or antigen-binding fragment thereof. In some embodiments, at least one of the two or more split doses is administered at least about 0.5 days after the immediately preceding dose. In some embodiments, at least one of the two or more split doses is administered about 1 day after the immediately preceding dose. In some embodiments, the anti-PD-1 antibody or antigen-binding fragment thereof is administered to the subject in one or more doses of about 0.1 mg/kg to about 20 mg/kg of body weight of the subject. In some embodiments, the anti-PD-1 antibody or antigen-binding fragment thereof is administered to the subject in one or more doses of about 1 mg to about 1500 mg. In some embodiments, the anti-PD-1 antibody or antigen-binding fragment thereof is administered to the subject once a day, once every two days, once every three days, once every five days, once every week, once every two weeks, once every three weeks, once every four weeks, once every five weeks or once every six weeks. In some embodiments, at least one of the one or more doses of the anti-PD-1 antibody or antigen-binding fragment thereof is administered 0.5 to 12 weeks after the immediately preceding dose. In some embodiments, at least one of the one or more doses of the anti-PD-1 antibody or antigen-binding fragment thereof includes a dose having a greater amount of the anti-PD-1 antibody or antigen-binding fragment thereof than the immediately preceding dose thereof.

In some embodiments, the bispecific antibody or antigen-binding fragment thereof or the anti-PD-1 antibody or antigen-binding fragment thereof are administered to the subject intravenously, subcutaneously, or intraperitoneally.

In some embodiments, the subject has cytokine release syndrome. In some embodiments, the tumor includes a B-cell cancer. In some embodiments, the B-cell cancer is selected from Hodgkin's lymphoma, non-Hodgkin's lymphoma, follicular lymphoma, small lymphocytic lymphoma, lymphoplasmacytoid lymphoma, marginal zone lymphoma, mantle cell lymphoma, diffuse large B-cell lymphoma, B-cell lymphomas, lymphomatoid granulomatosis, Burkitt's lymphoma, acute lymphoblastic leukemia, hairy cell leukemia, and B-cell chronic lymphocytic leukemia.

In some embodiments, the subject is resistant to, inadequately responsive to, or relapsed after prior therapy. In some embodiments, the subject has been treated with prior anti-CD20 therapy. In some embodiments, the anti-CD20 therapy includes an anti-CD20 antibody. In some embodiments, the treatment produces a therapeutic effect selected from delay in tumor growth, reduction in tumor cell number, tumor regression, increase in survival, partial response, and complete response.

In some embodiments, the treatment leads to an effect selected from reduced cytokine release, reduced release of IL-2, IL-6, IL-10, TNF-α and/or IFN-γ, reduced administration of dexamethasone, corticosteroids or an analgesic, reduced number of immune related adverse events, and reduced number of ≥Grade 3 adverse events.

In some embodiments, tumor growth is delayed by at least 10 days as compared to an untreated subject. In some embodiments, the tumor growth is inhibited by at least 50% as compared to an untreated subject. In some embodiments, the tumor growth is inhibited by at least 50% as compared to a subject administered with either the bispecific antibody or antigen-binding fragment thereof or the anti-PD-1 antibody or antigen-binding fragment thereof as monotherapy.

In some embodiments, the method further includes administering to the subject a third therapeutic agent or therapy. In some embodiments, the third therapeutic agent or therapy is selected from radiation, surgery, a chemotherapeutic agent, a cancer vaccine, a PD-L1 inhibitor, a LAG-3 inhibitor, a CTLA-4 inhibitor, a TIM3 inhibitor, a BTLA inhibitor, a TIGIT inhibitor, a CD47 inhibitor, a CD28 activator, a CD38 inhibitor, a GITR agonist, an indoleamine-2,3-dioxygenase (IDO) inhibitor, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an antibody to a tumor-specific antigen, Bacillus Calmette-Guerin vaccine, granulocyte-macrophage colony-stimulating factor, a cytotoxin, an interleukin 6 receptor (IL-6R) inhibitor, an interleukin 4 receptor (IL-4R) inhibitor, an IL-10 inhibitor, IL-2, IL-7, IL-12, IL-21, IL-15, an antibody-drug conjugate, an oncolytic virus, an anti-inflammatory drug, a dietary supplement, and combinations thereof.

In some embodiments, the first antigen-binding arm of the bispecific antibody or antigen-binding fragment thereof includes three heavy chain CDRs (A-HCDR1, A-HCDR2, and A-HCDR3) and three light chain CDRs (LCDR1, LCDR2, and LCDR3), and wherein A-HCDR1 includes the amino acid sequence of SEQ ID NO: 14; A-HCDR2 includes the amino acid sequence of SEQ ID NO: 15; A-HCDR3 includes the amino acid sequence of SEQ ID NO: 16; LCDR1 includes the amino acid sequence of SEQ ID NO: 17; LCDR2 includes the amino acid sequence of SEQ ID NO: 18; and LCDR3 includes the amino acid sequence of SEQ ID NO: 19. In some embodiments, the first antigen-binding arm of the bispecific antibody or antigen-binding fragment thereof includes a heavy chain variable region (A-HCVR) having the amino acid sequence of SEQ ID NO: 11 and a light chain variable region (LCVR) having the amino acid sequence of SEQ ID NO: 12.

In some embodiments, the second antigen-binding arm of the bispecific antibody or antigen-binding fragment thereof includes three heavy chain CDRs (B-HCDR1, B-HCDR2, and B-HCDR3) and three light chain CDRs (LCDR1, LCDR2, and LCDR3), and wherein B-HCDR1 includes the amino acid sequence of SEQ ID NO: 20; B-HCDR2 includes the amino acid sequence of SEQ ID NO: 21; B-HCDR3 includes the amino acid sequence of SEQ ID NO: 22; LCDR1 includes the amino acid sequence of SEQ ID NO: 17; LCDR2 includes the amino acid sequence of SEQ ID NO: 18; and LCDR3 includes the amino acid sequence of SEQ ID NO: 19. In some embodiments, the second antigen-binding arm of the bispecific antibody or antigen-binding fragment thereof includes a heavy chain variable region (B-HCVR) having the amino acid sequence of SEQ ID NO: 13 and a light chain variable region (LCVR) having the amino acid sequence of SEQ ID NO: 12.

In some embodiments, the bispecific antibody includes a first heavy chain including the HCVR of the first antigen-binding domain, a second heavy chain including the HCVR of the second antigen-binding domain, and a common light chain including the LCVR of the first and second antigen-binding domains, wherein the first heavy chain includes the amino acid sequence of SEQ ID NO: 23. In some embodiments, the bispecific antibody includes a first heavy chain including the HCVR of the first antigen-binding domain, a second heavy chain including the HCVR of the second antigen-binding domain, and a common light chain including the LCVR of the first and second antigen-binding domains, wherein the second heavy chain includes the amino acid sequence of SEQ ID NO: 25. In some embodiments, the bispecific antibody includes a first heavy chain including the HCVR of the first antigen-binding domain, a second heavy chain including the HCVR of the second antigen-binding domain, and a common light chain including the LCVR of the first and second antigen-binding domains, wherein the light chain includes the amino acid sequence of SEQ ID NO: 24.

In some embodiments, the bispecific antibody is odronextamab.

In some embodiments, the anti-PD-1 antibody or antigen-binding fragment thereof includes three heavy chain complementarity determining regions (HCDR1, HCDR2, and HCDR3) and three light chain complementarity determining regions (LCDR1, LCDR2, and LCDR3), and wherein HCDR1 includes the amino acid sequence of SEQ ID NO: 3; HCDR2 includes the amino acid sequence of SEQ ID NO: 4; HCDR3 includes the amino acid sequence of SEQ ID NO: 5; LCDR1 includes the amino acid sequence of SEQ ID NO: 6; LCDR2 includes the amino acid sequence of SEQ ID NO: 7; and LCDR3 includes the amino acid sequence of SEQ ID NO: 8. In some embodiments, the anti-PD-1 antibody or antigen-binding fragment thereof includes a heavy chain variable region (HCVR) having the amino acid sequence of SEQ ID NO: 1 and a light chain variable region (LCVR) having the amino acid sequence of SEQ ID NO: 2. In some embodiments, the anti-PD-1 antibody includes a heavy chain having the amino acid sequence of SEQ ID NO: 9 and a light chain having the amino acid sequence of SEQ ID NO: 10.

In some embodiments, the anti-PD-1 antibody is cemiplimab.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the disclosed subject matter will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1A shows an example engineered reporter assay to evaluate odronextamab (REGN1979)+cemiplimab (REGN2810), wherein titrations of odronextamab ranged from 500 nM to 0.05 pM.

FIG. 1B shows an example engineered reporter assay to evaluate odronextamab (REGN1979)+cemiplimab (REGN2810), wherein odronextamab stimulates AP1-Luc activity from T-cells incubated with WSU-DLCL2 cells.

FIG. 1C shows an example engineered reporter assay to evaluate odronextamab (REGN1979)+cemiplimab (REGN2810), wherein odronextamab stimulation of AP1-Luc in T-cells was significantly decreased in the presence of PD-L1 on WSU-DLCL2, which facilitated PD1 activation and repression of T-cell/AP1-Luc activity.

FIG. 2A is a first part of a schematic (continuing to FIG. 2B and FIG. 2C) showing a primary CD3+ T-cell assay to evaluate odronextamab+cemiplimab.

FIG. 2B is a second part of a schematic (beginning in FIG. 2A and continuing to FIG. 2C) showing a primary CD3+ T-cell assay to evaluate odronextamab+cemiplimab.

FIG. 2C is a third part of a schematic (beginning in FIG. 2A and FIG. 2B) showing a primary CD3+ T-cell assay to evaluate odronextamab+cemiplimab.

FIGS. 3A, 3B, 3C, 3D, 3E, 3F, and 3G are graphs showing the results from a odronextamab+cemiplimab lead-in dosing assay, in which T-cells exposed to a lead-in dose of odronextamab exhibited cytokine rescue through cemiplimab treatment.

FIG. 3A is a graph showing the results from a odronextamab+cemiplimab lead-in dosing assay, in which T-cells exposed to a lead-in dose of odronextamab exhibited cytokine rescue through cemiplimab treatment, wherein the graph shows IL-2 release (as measured by relative fluorescence unit or RFU) in response to a titration of cemiplimab in unstimulated T-cells with WSU-DLCL2/PD-L1.

FIG. 3B is a graph showing the results from a odronextamab+cemiplimab lead-in dosing assay, in which T-cells exposed to a lead-in dose of odronextamab exhibited cytokine rescue through cemiplimab treatment, wherein the graph shows IL-2 release in response to a titration of cemiplimab in T-cells pre-stimulated with an initial dose of 1.3 nM odronextamab.

FIG. 3C is an enhanced view of FIG. 3B.

FIG. 3D is a graph showing IFNγ release in response to a titration of cemiplimab in unstimulated T-cells.

FIG. 3E is a graph showing IFNγ release in response to a titration of cemiplimab in T-cells pre-stimulated with 1.3 nM odronextamab.

FID. 3F is an enhanced view of FIG. 3E.

FIG. 3G is a set of graphs showing PD-1 expression levels in unstimulated and pre-stimulated T-cells.

FIG. 4 is a set of graphs showing that cytokine response from pre-stimulation in response to blocking of CTLA-4 and LAG-3.

FIG. 5 is a set of graphs showing T-cell cytotoxicity of unstimulated T-cells and T-cells stimulated with 1.3 nM odronextamab.

FIG. 6A shows a quantitative systems pharmacology (QSP) modeling framework that captures disease-lymphocyte-drug interactions related to the odronextamab mechanism of action, wherein odronextamab binds to CD20 on B-cells and CD3 on T cells to form the synapse.

FIG. 6B shows a QSP modeling framework that captures disease-lymphocyte-drug interactions related to T cell activation, wherein odronextamab binds to T cells and B-cells, and activates T cells, which enhances T cell mediated tumor cytotoxicity.

FIG. 6C shows a QSP modeling framework that captures disease-lymphocyte-drug interactions related to an IL-6 dynamic model, wherein cemiplimab regulates cytokine production caused by synapse formation, and increases release of IL-6 into the systemic circulation. [IL, interleukin]

FIG. 6D shows a QSP modeling framework that captures disease-lymphocyte-drug interactions related to the cemiplimab mechanism of action, wherein cemiplimab promotes T cell activation.

FIG. 6E shows a QSP modeling framework that captures disease-lymphocyte-drug interactions related to the odronextamab/cemiplimab combination mechanism of action, wherein odronextamab/cemiplimab combination enhances T cell activation to kill tumor cells.

FIG. 6F shows a QSP modeling framework that captures disease-lymphocyte-drug interactions related to the PD-1 regulatory model, wherein PD-1 expression level on activated T cells is a function of programmed cell death 1 (PDCD1) gene regulation. [TCR, T cell receptor]

FIG. 7 shows a representative example of an odronextamab monotherapy regimen (1, 6, 12 mg QW; Weeks 1-36). Arrows represent treatment with odronextamab. [QW, once weekly]

FIG. 8 shows a representative example of an odronextamab/cemiplimab combination therapy regimen (odronextamab 1, 6, 12 mg QW/cemiplimab 3 mg/kg Q2W). Arrows in upper row represent treatment with odronextramab; arrows in lower row represent treatment with cemiplimab. [QW, once weekly; Q2W, once every two weeks]

FIG. 9A shows a comparison of simulated (line) and observed (dot) IL-6 concentrations over time under odronextamab monotherapy and odronextamab/cemiplimab combination therapy in the QSP model in Example 2.

FIG. 9B shows another comparison of simulated (line) and observed (dot) IL-6 concentrations over time under odronextamab monotherapy and odronextamab/cemiplimab combination therapy in the QSP model in Example 2. The model predicts that IL-6 peaks following odrnoextamab/cemiplimab combination therapy were higher than those with odronextamab monotherapy.

FIG. 10 is a graph showing the impact of varying cemiplimab starting time on IL-6 levels as predicted by the QSP model in Example 2 based on odronextamab doses of 1, 20, or 160 mg QW with cemiplimab dosing (3 mg/kg Q2W) at various starting times. The model predicts that delaying cemiplimab dosing time could attenuate IL-6 peak levels.

FIG. 11 shows simulated cytokine release (IL-6) profiles based on an odronextamab monotherapy 4-week lead-in period followed by cemiplimab initiation at 3, 30, and 350 mg dose levels from Week 5 as predicted by the QSP model in Example 2. The model predicts that when starting cemiplimab dosing from Week 5, IL-6 levels are similar to that of odronextamab monotherapy regardless of cemiplimab dose levels.

### DETAILED DESCRIPTION

Targeted therapies for cancer hold the potential for specifically identifying and destroying cancer cells, while minimizing adverse side effects. However, these novel therapies present unique challenges for determining a dosing regimen that will be safe for patients and avoid unwanted immune cell activation. This disclosure is based, at least in part, on the unexpected discovery that pretreatment of the T-cells with an anti-CD20/anti-CD3 bispecific antibody (odronextamab) and delaying exposure to an anti-PD-1 antibody (cemiplimab) led to a marked decrease in cytokine (e.g., IL-2) release upon exposure to the combination of odronextamab plus cemiplimab, compared to control T-cells that had not undergone odronextamab pretreatment. This reduction in cytokine levels seen after odronextamab pretreatment supports the conclusion that initiating odronextamab before adding cemiplimab can help to significantly mitigate cytokine release syndrome (CRS).

The disclosed methods, which generally include administering to a subject with cancer a bispecific anti-CD20/anti-CD3 antibody for a lead-in period before administering to the subject an anti-PD-1 antibody, achieve a dual purpose by effectively treating or inhibiting the growth of a tumor (e.g., a B-cell cancer) while also avoiding or minimizing (i.e., achieving a lower incidence or severity of) the immune-related adverse event CRS.

Methods of Treatment

In one aspect, this disclosure provides methods for treating, ameliorating or reducing the severity of at least one symptom or indication, or inhibiting the growth of a cancer in a subject. The method comprises: (i) administering to a subject in need thereof a therapeutically effective amount of a bispecific anti-CD20/anti-CD3 antibody or antigen-binding fragment thereof comprising a first antigen-binding arm that specifically binds CD20 and a second antigen-binding arm that specifically binds CD3, followed by (ii) administering to the subject in need thereof an anti-PD-1 antibody or antigen-binding fragment thereof that specifically binds PD-1. Further, in this aspect, the method also ameliorates CRS in the subject.

In another aspect, this disclosure provides a method of ameliorating or reducing the severity of at least one symptom or indication of CRS in a subject with a tumor. The method comprises: (i) administering to a subject in need thereof a therapeutically effective amount of a bispecific anti-CD20/anti-CD3 antibody or antigen-binding fragment thereof comprising a first antigen-binding arm that specifically binds CD20 and a second antigen-binding arm that specifically binds CD3, followed by (ii) administering to the subject in need thereof an anti-PD-1 antibody or antigen-binding fragment thereof that specifically binds PD-1.

In some embodiments, the methods comprise administering to a subject in need thereof one or more doses of an anti-CD20/anti-CD3 bispecific antibody or antigen-binding fragment thereof before administering to the subject one or more doses of an anti-PD-1 antibody or antigen-binding fragment thereof. In administering one or more doses of the anti-PD-1 antibody or antigen-binding fragment thereof, the anti-PD-1 antibody or antigen-binding fragment thereof can be administered in combination with one or more doses of the anti-CD20/anti-CD3 bispecific antibody or antigen-binding fragment thereof.

As used herein, the terms “treat,” “treating,” or the like, mean to alleviate symptoms, eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, and/or to increase duration of survival of the subject.

As used herein, the expression “a subject in need thereof” means a human or non-human mammal that exhibits one or more symptoms or indications of cancer, and/or who has been diagnosed with cancer, including a B-cell cancer and who needs treatment for the same. In some embodiments, the term “subject” is used interchangeably with the term “patient.” For example, a human subject may be diagnosed with a primary or a metastatic tumor and/or with one or more symptoms or indications including, but not limited to, enlarged lymph node(s), swollen abdomen, chest pain/pressure, unexplained weight loss, fever, night sweats, persistent fatigue, loss of appetite, enlargement of spleen, itching. The expression includes subjects with primary or established B-cell tumors. In specific embodiments, the expression includes human subjects that have and need treatment for a B-cell malignancy, e.g., Hodgkin's lymphoma, non-Hodgkin's lymphoma, follicular lymphoma, small lymphocytic lymphoma, lymphoplasmacytoid lymphoma, marginal zone lymphoma, mantle cell lymphoma, diffuse large B-cell lymphoma, B-cell lymphomas, lymphomatoid granulomatosis, Burkitt's lymphoma, acute lymphoblastic leukemia, hairy cell leukemia, and B-cell chronic lymphocytic leukemia. In a further embodiment, the expression includes persons with a pathologic subtype of a B-cell cancer. In other specific embodiments, the expression includes subjects with CD20+ tumors (e.g., a tumor with CD20 expression as determined by flow cytometry on ≥20% of leukemic lymphoblasts). In certain embodiments, the expression “a subject in need thereof” includes patients with a B-cell cancer that is resistant to or refractory to or is inadequately controlled by prior therapy (e.g., treatment with a conventional anti-cancer agent). For example, the expression includes subjects who have been treated with a CD20 inhibitor (e.g., rituximab), chemotherapy, or an immune-modulating agent such as a blocker of CTLA-4, 4-1BB, LAG3 or OX-40. The expression also includes subjects with a B-cell malignancy for which conventional anti-cancer therapy is inadvisable, for example, due to toxic side effects. For example, the expression includes patients who have received one or more cycles of chemotherapy with toxic side effects. In certain embodiments, the expression “a subject in need thereof” includes patients with a B-cell malignancy that has been treated but which has subsequently relapsed or metastasized. For example, patients with a B-cell malignancy that may have received treatment with one or more anti-cancer agents leading to tumor regression; however, subsequently have relapsed with cancer resistant to the one or more anti-cancer agents (e.g., chemotherapy-resistant cancer) are treated with the methods of the present disclosure.

The expression “a subject in need thereof” also includes subjects who are at risk of developing a B-cell cancer, e.g., persons with a family history of lymphoma, persons with a past history of Epstein-Barr infections such as infectious mononucleosis, or persons with an immune system compromised due to HIV infection or due to immunosuppressive medications.

In certain embodiments, the methods of the present disclosure may be used to treat patients that show elevated levels of one or more cancer-associated biomarkers (e.g., PD-L1, CD20, beta-2-microglobulin, lactate dehydrogenase, BCR-ABL fusion gene, ALK gene rearrangement). For example, the methods comprise administering a therapeutically effective amount of a bispecific anti-CD20/anti-CD3 antibody or antigen-binding fragment thereof in combination with an anti-PD-1 antibody or antigen-binding fragment thereof to a patient with an elevated level of PD-L1 and/or CD20. In some embodiments, the methods comprise administering a bispecific anti-CD20/anti-CD3 antibody or antigen-binding fragment thereof a patient with an elevated level of PD-L1 and/or CD20 followed by administering an anti-PD-1 antibody or antigen-binding fragment thereof to the patient.

In certain embodiments, the methods of the present disclosure may be used to ameliorate or reduce the severity of at least one symptom or indication of CRS in a subject with a tumor. CRS is a systemic inflammatory response that can be triggered by a variety of factors, including certain drugs. T cell-activating cancer immunotherapies carry a particularly high risk of CRS, which is usually due to on-target effects induced by binding of a bispecific antibody or chimeric antigen receptor CAR-T cell to its antigen and subsequent activation of bystander immune cells and non-immune cells, such as endothelial cells. Activation of the bystander cells results in the massive release of a range of cytokines. IL-6, IL-10, and interferon (IFN)-γ are among the core cytokines that are consistently found to be elevated in serum of patients with CRS. With T cell-activating therapies directed against tumor cells, CRS is triggered by the massive release of IFN-γ by activated T cells or the tumor cells themselves. Secreted IFN-γ induces activation of other immune cells, most importantly macrophages, which in turn produce excessive amounts of additional cytokines such as IL-6, TNF-α, and IL-10.

In certain embodiments, the methods of the present disclosure are used in a subject with a B-cell cancer. The terms “tumor,” “tumor cells,” “cancer,” and “malignancy” are interchangeably used herein. The term “B-cell cancer,” as used herein, refers to tumors of white blood cells known as B-lymphocytes and includes leukemias (located in the blood) and lymphomas (located in the lymph nodes). The present disclosure includes methods to treat both leukemias and lymphomas. In certain embodiments, B-cell cancer includes, but is not limited to, Hodgkin's lymphoma, non-Hodgkin's lymphoma, follicular lymphoma, small lymphocytic lymphoma, lymphoplasmacytoid lymphoma, marginal zone lymphoma, mantle cell lymphoma, diffuse large B-cell lymphoma, B-cell lymphomas, lymphomatoid granulomatosis, Burkitt's lymphoma, acute lymphoblastic leukemia, hairy cell leukemia, B-cell chronic lymphocytic leukemia, as well as the pathologic subtypes thereof. B-cell lymphomas are typically divided into low and high grade, typically corresponding to indolent (slow-growing) lymphomas and aggressive lymphomas, respectively. The present disclosure includes methods to treat both indolent and aggressive lymphomas.

According to certain embodiments, the present disclosure includes methods for treating, delaying, or inhibiting the growth of a tumor. In certain embodiments, the present disclosure includes methods to promote tumor regression. In certain embodiments, the present disclosure includes methods to reduce tumor cell load or to reduce tumor burden. In certain embodiments, the present disclosure includes methods to prevent tumor recurrence.

The methods, according to this aspect of the present disclosure, comprise sequentially administering a therapeutically effective amount of a bispecific anti-CD20/anti-CD3 antibody or antigen-binding fragment thereof in combination with a therapeutically effective amount of an anti-PD-1 antibody or antigen-binding fragment thereof to a subject in need thereof, wherein each antibody is administered to the subject in multiple doses, e.g., as part of a specific therapeutic dosing regimen. For example, the therapeutic dosing regimen may comprise administering one or more doses of a therapeutically effective amount of a bispecific anti-CD20/anti-CD3 antibody or antigen-binding fragment thereof, wherein the one or more doses of the bispecific antibody or antigen-binding fragment thereof are administered to the subject at a frequency of about once a day, once every two days, once every three days, once every four days, once every five days, once every six days, once a week, once every two weeks, once every three weeks, once every four weeks, once a month, once every two months, once every three months, once every four months, or less frequently. In certain embodiments, at least one dose of the anti-CD20/anti-CD3 antibody or antigen-binding fragment thereof is administered in more than 1 fractions, e.g., in 2-5 fractions (“split dosing”) within the given dosing period. The anti-CD20/anti-CD3 bispecific antibody or antigen-binding fragment thereof may be administered in split doses to reduce or eliminate the cytokine “spikes” induced in response to administration of the antibody. Cytokine spikes refer to the clinical symptoms of the cytokine release syndrome (CRS) or “cytokine storm” and infusion-related reactions, seen in patients administered anti-CD20 antibodies.

In certain embodiments, the one or more doses of anti-CD20/anti-CD3 antibody or antigen-binding fragment thereof are administered in combination with one or more doses of an anti-PD-1 antibody or antigen-binding fragment thereof to the subject at a frequency of about once a day, once every two days, once every three days, once every four days, once every five days, once every six days, once a week, once every two weeks, once every three weeks, once every four weeks, once a month, once every two months, once every three months, once every four months, or less frequently.

In certain embodiments, the methods of the present disclosure comprise administering one or more doses of a bispecific anti-CD20/anti-CD3 antibody or antigen-binding fragment thereof in combination with one or more doses of anti-PD-1 antibody or antigen-binding fragment thereof to a subject in need thereof, wherein a dose of the bispecific antibody is administered as split doses, or in more than 1 fractions, e.g., as 2 fractions, as 3 fractions, as 4 fractions or as 5 fractions within the given dosing period. In certain embodiments, a dose of the bispecific antibody is split into 2 or more fractions, wherein each fraction comprises an amount of the antibody equal to the other fractions.

In some embodiments, the one or more doses of the bispecific antibody or antigen-binding fragment thereof is administered to the subject at least about 1 week prior to administering the one or more dose of the anti-PD-1 antibody or antigen-binding fragment thereof. In some embodiments, the first dose of the bispecific antibody or antigen-binding fragment thereof is administered to the subject about 1 week, 2 weeks, 3 weeks, 4 weeks, 5 weeks, 6 weeks, 7 weeks, or 8 weeks prior to administering the first dose of the anti-PD-1 antibody or antigen-binding fragment thereof. In some embodiments, the first dose of the bispecific antibody or antigen-binding fragment thereof is administered to the subject about 5 weeks (e.g., 5 weeks) prior to administering the first dose of the anti-PD-1 antibody or antigen-binding fragment thereof.

In some embodiments, the bispecific antibody or antigen-binding fragment thereof is administered to the subject in one or more doses of about 0.1 mg/kg to about 15 mg/kg of body weight of the subject. In some embodiments, the bispecific antibody or antigen-binding fragment thereof is administered to the subject in one or more doses of about 1 mg to about 800 mg.

In some embodiments, at least one of the one or more doses of the bispecific antibody or antigen-binding fragment thereof comprises a dose having a greater amount of the bispecific antibody or antigen-binding fragment thereof than the immediately preceding dose thereof.

In some embodiments, at least one of the one or more doses of the bispecific antibody or antigen-binding fragment thereof is administered in two or more split doses. In some embodiments, at least one of the two or more split doses comprises the identical amount of the bispecific antibody or antigen-binding fragment thereof. In some embodiments, at least one of the two or more split doses is administered at least about 0.5 days after the immediately preceding dose. In some embodiments, each of the two or more split doses is administered about 1 day after the immediately preceding dose.

For example, a dose of anti-CD20/anti-CD3 antibody comprising 1 mg of anti-CD20/anti-CD3 antibody or antigen-binding fragment thereof may be administered once a week, wherein the dose is administered in 2 fractions within the week, each fraction comprising 0.5 mg. In some embodiments, a dose of anti-CD20/anti-CD3 antibody comprising 1 mg of anti-CD20/anti-CD3 antibody or antigen-binding fragment thereof may be administered once a week, wherein the dose is administered in 2 fractions on two consecutive days respectively, each fraction comprising 0.5 mg. In some embodiments, a dose of anti-CD20/anti-CD3 antibody comprising 4 mg of anti-CD20/anti-CD3 antibody or antigen-binding fragment thereof may be administered once a week, wherein the dose is administered in 2 fractions within the week, each fraction comprising 2 mg. In some embodiments, a dose of anti-CD20/anti-CD3 antibody comprising 20 mg of anti-CD20/anti-CD3 antibody or antigen-binding fragment thereof may be administered once a week, wherein the dose is administered in 2 fractions within the week, each fraction comprising 10 mg. In some embodiments, a dose of anti-CD20/anti-CD3 antibody comprising 20 mg of anti-CD20/anti-CD3 antibody or antigen-binding fragment thereof may be administered once a week, wherein the dose is administered in 2 fractions on two consecutive days respectively, each fraction comprising 10 mg. In some embodiments, a dose of anti-CD20/anti-CD3 antibody comprising 80 mg of anti-CD20/anti-CD3 antibody or antigen-binding fragment thereof may be administered once a week, wherein the dose is administered in 2 fractions within the week, each fraction comprising 40 mg. In some embodiments, a dose of anti-CD20/anti-CD3 antibody comprising 80 mg of anti-CD20/anti-CD3 antibody or antigen-binding fragment thereof may be administered once a week, wherein the dose is administered in 2 fractions on two consecutive days respectively, each fraction comprising 40 mg. In some embodiments, a dose of anti-CD20/anti-CD3 antibody comprising 320 mg of anti-CD20/anti-CD3 antibody or antigen-binding fragment thereof may be administered once a week, wherein the dose is administered in 2 fractions within the week, each fraction comprising 160 mg. In some embodiments, a dose of anti-CD20/anti-CD3 antibody comprising 320 mg of anti-CD20/anti-CD3 antibody or antigen-binding fragment thereof may be administered once a week, wherein the dose is administered in 2 fractions on two consecutive days respectively, each fraction comprising 160 mg.

In certain embodiments, a dose of the bispecific antibody is administered split into 2 or more fractions, wherein the fractions comprise unequal amounts of the antibody, e.g., more than or less than the first fraction. For example, a dose of anti-CD20/anti-CD3 antibody comprising 360 mg may be administered once a week, wherein the dose is administered in 2 fractions within the week, wherein the first fraction comprises 200 mg and the second fraction comprises 160 mg. As another example, a dose of anti-CD20/anti-CD3 antibody comprising 360 mg may be administered once in 2 weeks, wherein the dose is administered in 3 fractions within the 2-week period, wherein the first fraction comprises 120 mg, the second fraction comprises 120 mg, and the third fraction comprises 120 mg.

In some embodiments, the anti-PD-1 antibody or antigen-binding fragment thereof is administered to the subject in one or more doses of about 0.1 mg/kg to about 20 mg/kg of body weight of the subject. In some embodiments, the anti-PD-1 antibody or antigen-binding fragment thereof is administered to the subject in one or more doses of about 1 mg to about 800 mg.

In some embodiments, the anti-PD-1 antibody or antigen-binding fragment thereof is administered to the subject once a day, once every two days, once every three days, once every five days, once every week, once every two weeks, or once every three weeks. In some embodiments, each dose of the one or more doses of the anti-PD-1 antibody or antigen-binding fragment thereof is administered 0.5 to 12 weeks after the immediately preceding dose.

In some embodiments, at least one of the one or more doses of the anti-PD-1 antibody or antigen-binding fragment thereof comprises a dose having a greater amount of the anti-PD-1 antibody or antigen-binding fragment thereof than the immediately preceding dose thereof.

An example of a dosing regimen contemplated by the present disclosure is as follows:

| | |
|---|---|
| Week 1 | 1 mg odronextamab administered on 2 consecutive days, 0.5 |
| | mg/day |
| Week 2 | 4 mg odronextamab administered on 2 consecutive days, 2 |
| | mg/day |
| Week 3 | 20 mg odronextamab administered on 2 consecutive days, 10 |
| | mg/day |
| Week 4 | 80 mg odronextamab administered on 2 consecutive days, 40 |
| | mg/day |
| Weeks 5 | 80 mg odronextamab administered in one dose |
| to 12-16 | |
| Weeks | 160 mg odronextamab administered once every 2 weeks |
| 13-17 | |
| Week 5 | in combination with odronextamab (above), administer |
| | cemiplimab - initially 3 mg, then 30 mg, then 350 mg. Either |
| | once every two weeks (Q2W) or once every four weeks |
| | (Q4W). |

In certain embodiments, the present disclosure includes methods to inhibit, retard, or stop tumor metastasis or tumor infiltration into peripheral organs. The methods according to this aspect comprise administering a therapeutically effective amount of a bispecific anti-CD20/anti-CD3 antibody or antigen-binding fragment thereof in combination with an anti-PD-1 antibody or antigen-binding fragment thereof to a subject in need thereof. In specific embodiments, the present disclosure provides methods for increased anti-tumor efficacy or increased tumor inhibition.

The methods, according to this aspect of the present disclosure, comprise administering to a subject with a B-cell cancer a therapeutically effective amount of a bispecific anti-CD20/anti-CD3 antibody or antigen-binding fragment thereof prior to administering a therapeutically effective amount of an anti-PD-1 antibody or antigen-binding fragment thereof, wherein the anti-CD20/anti-CD3 antibody or antigen-binding fragment thereof may be administered about 1 day, more than 1 day, more than 2 days, more than 3 days, more than 4 days, more than 5 days, more than 6 days, more than 7 days, more than 8 days, more than 10 days, more than 1 week, more than 2 weeks, more than 3 weeks, more than 4 weeks, more than 5 weeks, more than 6 weeks, more than 7 weeks, or more than 8 weeks prior to the anti-PD-1 antibody or antigen-binding fragment thereof. In certain embodiments, the methods provide for increased tumor inhibition, e.g., by about 20%, more than 20%, more than 30%, more than 40% more than 50%, more than 60%, more than 70% or more than 80% as compared to a monotherapy by the bispecific antibody or the anti-PD-1 antibody.

In certain embodiments, the methods of the present disclosure comprise administering a therapeutically effective amount of a bispecific anti-CD20/anti-CD3 antibody or antigen-binding fragment thereof in combination with an anti-PD-1 antibody or antigen-binding fragment thereof to a subject with a B-cell cancer. For example, the methods comprise administering a therapeutically effective amount of a bispecific anti-CD20/anti-CD3 antibody or antigen-binding fragment thereof prior to administering an anti-PD-1 antibody or antigen-binding fragment thereof to a subject with a B-cell cancer. In specific embodiments, the B-cell cancer is Hodgkin's lymphoma or non-Hodgkin's lymphoma. In further embodiments, the B-cell cancer is indolent or aggressive. In certain embodiments, the subject is not responsive to prior therapy or has relapsed after prior therapy.

In certain embodiments, the methods of the present disclosure further comprise administering a bispecific anti-CD20/anti-CD3 antibody or antigen-binding fragment thereof in combination with an anti-PD-1 antibody to a subject with a CD20+B-cell cancer. In certain embodiments, the methods of the present disclosure comprise administering a therapeutically effective amount of a bispecific anti-CD20/anti-CD3 antibody or antigen-binding fragment thereof prior to administering an anti-PD-1 antibody or antigen-binding fragment thereof to a subject with a CD20+B-cell cancer. In specific embodiments, the B-cell cancer is acute lymphoblastic leukemia or chronic lymphocytic leukemia. In further embodiments, the B-cell cancer is indolent or aggressive. In certain embodiments, the subject is not responsive to prior therapy or has relapsed after prior therapy (e.g., with an anti-CD20 inhibitor such as rituximab).

In certain embodiments, the methods of the present disclosure comprise administering a bispecific anti-CD20/anti-CD3 antibody or antigen-binding fragment thereof in combination with an anti-PD-1 antibody or antigen-binding fragment thereof to a subject in need thereof as a “first line” treatment (e.g., initial treatment). In other embodiments, a bispecific anti-CD20/anti-CD3 antibody or antigen-binding fragment thereof in combination with an anti-PD-1 antibody or antigen-binding fragment thereof administered as a “second line” treatment (e.g., after prior therapy). For example, a bispecific anti-CD20/anti-CD3 antibody or antigen-binding fragment thereof in combination with an anti-PD-1 antibody or antigen-binding fragment thereof is administered as a “second line” treatment to a subject that has relapsed after prior therapy with, e.g., chemotherapy or rituximab.

In certain embodiments, the methods of the present disclosure are used to treat a patient with a minimum residual disease (MRD)-positive disease. MRD refers to small numbers of cancer cells that remain in the patient during or after treatment, wherein the patient may or may not show symptoms or signs of the disease. Such residual cancer cells, if not eliminated, frequently lead to relapse of the disease. The present disclosure includes methods to inhibit and/or eliminate residual cancer cells in a patient upon MRD testing. MRD may be assayed according to methods known in the art (e.g., MRD flow cytometry). The methods, according to this aspect of the present disclosure, comprise administering a bispecific anti-CD20/anti-CD3 antibody or antigen-binding fragment thereof to a subject in need thereof in combination with an anti-PD-1 antibody or antigen-binding fragment thereof.

The methods of the present disclosure, according to certain embodiments, comprise administering to a subject a therapeutically effective amount of each of a bispecific anti-CD20/anti-CD3 antibody or antigen-binding fragment thereof and an anti-PD-1 antibody or antigen-binding fragment thereof in combination with a third therapeutic agent or therapy. The third therapeutic agent may be an agent selected from the group consisting of, e.g., radiation, chemotherapy, surgery, a cancer vaccine, a PD-L1 inhibitor (e.g., an anti-PD-L1 antibody), a LAG3 inhibitor (e.g., an anti-LAG3 antibody), a CTLA-4 inhibitor, a TIM3 inhibitor, a BTLA inhibitor, a TIGIT inhibitor, a CD47 inhibitor, an indoleamine-2,3-dioxygenase (IDO) inhibitor, a vascular endothelial growth factor (VEGF) antagonist, an Ang2 inhibitor, a transforming growth factor beta (TGFβ) inhibitor, an epidermal growth factor receptor (EGFR) inhibitor, an antibody to a tumor-specific antigen (e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9), an oncolytic virus, a vaccine (e.g., Bacillus Calmette-Guerin), granulocyte-macrophage colony-stimulating factor, a cytotoxin, a chemotherapeutic agent, an IL-6R inhibitor, an IL-4R inhibitor, an IL-10 inhibitor, a cytokine such as IL-2, IL-7, IL-12, IL-21, and IL-15, an anti-inflammatory drug such as corticosteroids, and non-steroidal anti-inflammatory drugs, a dietary supplement such as an antioxidants, and combinations thereof.

In certain embodiments, the antibodies may be administered in combination with therapy, including a chemotherapeutic agent, radiation, and surgery. In this context, the phrase ‘in combination with” means that the antibodies are administered to the subject at the same time as, just before, or just after administration of the third therapeutic agent. In certain embodiments, the third therapeutic agent is administered as a co-formulation with the antibodies. In a related embodiment, the present disclosure includes methods comprising administering a therapeutically effective amount of a bispecific anti-CD20/anti-CD3 antibody or antigen-binding fragment thereof in combination with an anti-PD-1 antibody or antigen-binding fragment thereof to a subject who is on a background anti-cancer therapeutic regimen. The background anti-cancer therapeutic regimen may comprise a course of administration of, e.g., a chemotherapeutic agent or radiation. The bispecific anti-CD20/anti-CD3 antibody or antigen-binding fragment thereof in combination with an anti-PD-1 antibody or antigen-binding fragment thereof may be added on top of the background anti-cancer therapeutic regimen. In some embodiments, the antibodies are added as part of a “background step-down” scheme, wherein the background anti-cancer therapy is gradually withdrawn from the subject over time (e.g., in a stepwise fashion) while the antibodies are administered to the subject at a constant dose, or at an increasing dose, or at a decreasing dose, over time.

In certain embodiments, the methods of the present disclosure comprise administering to a subject in need thereof a therapeutically effective amount of a bispecific anti-CD20/anti-CD3 antibody or antigen-binding fragment thereof in combination with a therapeutically effective amount of an anti-PD-1 antibody or antigen-binding fragment thereof, wherein administration of the antibodies leads to increased inhibition of tumor growth. In certain embodiments, tumor growth is inhibited by at least about 10%, about 20%, about 30%, about 40%, about 50%, about 60%, about 70% or about 80% as compared to an untreated subject or a subject administered with either antibody as monotherapy. In certain embodiments, the administration of a bispecific anti-CD20/anti-CD3 antibody or antigen-binding fragment thereof and/or an anti-PD-1 antibody or antigen-binding fragment thereof leads to increased tumor regression, tumor shrinkage and/or disappearance. In certain embodiments, the administration of a bispecific anti-CD20/anti-CD3 antibody or antigen-binding fragment thereof and/or an anti-PD-1 antibody or antigen-binding fragment thereof leads to delay in tumor growth and development, e.g., tumor growth may be delayed by about 3 days, more than 3 days, about 7 days, more than 7 days, more than 15 days, more than 1 month, more than 3 months, more than 6 months, more than 1 year, more than 2 years, or more than 3 years as compared to an untreated subject or a subject treated with either antibody as monotherapy. In certain embodiments, administration of a bispecific anti-CD20/anti-CD3 antibody or antigen-binding fragment thereof in combination with an anti-PD-1 antibody or antigen-binding fragment thereof prevents tumor recurrence and/or increases duration of survival of the subject, e.g., increases duration of survival by more than 15 days, more than 1 month, more than 3 months, more than 6 months, more than 12 months, more than 18 months, more than 24 months, more than 36 months, or more than 48 months than an untreated subject or a subject which is administered either antibody as monotherapy. In certain embodiments, administration of the antibodies in combination increases progression-free survival or overall survival.

In certain embodiments, administration of a bispecific anti-CD20/anti-CD3 antibody or antigen-binding fragment thereof in combination with an anti-PD-1 antibody or antigen-binding fragment thereof increases response and duration of response in a subject, e.g., by more than 2%, more than 3%, more than 4%, more than 5%, more than 6%, more than 7%, more than 8%, more than 9%, more than 10%, more than 20%, more than 30%, more than 40% or more than 50% over an untreated subject or a subject which has received either antibody as monotherapy. In certain embodiments, administration of a bispecific anti-CD20/anti-CD3 antibody or antigen-binding fragment thereof in combination with an anti-PD-1 antibody or antigen-binding fragment thereof to a subject with a B-cell cancer leads to complete disappearance of all evidence of tumor cells (“complete response”). In certain embodiments, administration of an anti-PD-1 antibody or antigen-binding fragment thereof and/or a bispecific anti-CD20/anti-CD3 antibody or antigen-binding fragment thereof to a subject with a B-cell cancer leads to at least 30% or more decrease in tumor cells or tumor size (“partial response”). In certain embodiments, administration of a bispecific anti-CD20/anti-CD3 antibody or antigen-binding fragment thereof and/or an anti-PD-1 antibody or antigen-binding fragment thereof to a subject with a B-cell cancer leads to complete or partial disappearance of tumor cells/lesions including new measurable lesions. Tumor reduction can be measured by any of the methods known in the art, e.g., X-rays, positron emission tomography (PET), computed tomography (CT), magnetic resonance imaging (MRI), cytology, histology, or molecular genetic analyses.

In certain embodiments, the combination of administered antibodies is safe and well-tolerated by a patient wherein there is no increase in an adverse side effect (e.g., increased CRS or increased T-cell activation) as compared to a patient administered with the bispecific antibody as monotherapy.

Anti-PD-1 Antibodies and Antigen-Binding Fragments Thereof

According to certain exemplary embodiments of the present disclosure, the methods comprise administering a therapeutically effective amount of an anti-PD-1 antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or VH) and a heavy chain constant region. The heavy chain constant region comprises three domains, CH1, CH2, and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL are composed of three CDRs and four FRs, arranged from amino-terminus to carboxy-terminus in the following order: FR1, CDR1, FR2, CDR2, FR3, CDR3, FR4. In different embodiments of the present disclosure, the FRs of the anti-IL-4R antibody (or antigen-binding portion thereof) may be identical to the human germline sequences or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L}, or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present disclosure using routine techniques available in the art. For example, the present disclosure includes methods comprising the use of bispecific antibodies wherein one arm of an immunoglobulin is specific for PD-1 or a fragment thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual-acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency, and geometry. (See, e.g., Kazane et al., J. Am. Chem. Soc. Epub: Dec. 4, 2012).

The antibodies used in the methods of the present disclosure may be human antibodies. The term “human antibody,” as used herein, is intended to include antibodies having variable and constant regions derived from human germline immunoglobulin sequences. The human antibodies of the present disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present disclosure may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see, e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

According to certain embodiments, the antibodies used in the methods of the present disclosure specifically bind PD-1. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” PD-1, as used in the context of the present disclosure, includes antibodies that bind PD-1 or portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human PD-1 may, however, have cross-reactivity to other antigens, such as PD-1 molecules from other (non-human) species.

According to certain exemplary embodiments of the present disclosure, the anti-PD-1 antibody, or antigen-binding fragment thereof comprises a heavy chain variable region (HCVR), light chain variable region (LCVR), and/or complementarity determining regions (CDRs) comprising any of the amino acid sequences of the anti-PD-1 antibodies as set forth in US Patent Publication No. 20150203579. In certain exemplary embodiments, the anti-PD-1 antibody or antigen-binding fragment thereof that can be used in the context of the methods of the present disclosure comprises the heavy chain complementarity determining regions (HCDRs) of a heavy chain variable region (HCVR) comprising the amino acid sequence of SEQ ID NO: 1 and the light chain complementarity determining regions (LCDRs) of a light chain variable region (LCVR) comprising the amino acid sequence of SEQ ID NO: 2. According to certain embodiments, the anti-PD-1 antibody or antigen-binding fragment thereof comprises three HCDRs (HCDR1, HCDR2, and HCDR3) and three LCDRs (LCDR1, LCDR2, and LCDR3), wherein the HCDR1 comprises the amino acid sequence of SEQ ID NO: 3; the HCDR2 comprises the amino acid sequence of SEQ ID NO: 4; the HCDR3 comprises the amino acid sequence of SEQ ID NO: 5; the LCDR1 comprises the amino acid sequence of SEQ ID NO: 6; the LCDR2 comprises the amino acid sequence of SEQ ID NO: 7; and the LCDR3 comprises the amino acid sequence of SEQ ID NO: 8. In yet other embodiments, the anti-PD-1 antibody or antigen-binding fragment thereof comprises an HCVR comprising SEQ ID NO: 1 and an LCVR comprising SEQ ID NO: 2. In certain embodiments, the methods of the present disclosure comprise the use of an anti-PD-1 antibody, wherein the antibody comprises a heavy chain comprising the amino acid sequence of SEQ ID NO: 9. In some embodiments, the anti-PD-1 antibody comprises a light chain comprising the amino acid sequence of SEQ ID NO: 10. An exemplary antibody comprising a HCVR comprising the amino acid sequence of SEQ ID NO: 1 and a LCVR comprising the amino acid sequence of SEQ ID NO: 2 is the fully human anti-PD-1 antibody known as REGN2810 (also known as cemiplimab, LIBTAYO®). According to certain exemplary embodiments, the methods of the present disclosure comprise the use of cemiplimab or a bioequivalent thereof.

The term “bioequivalent,” as used herein, refers to anti-PD-1 antibodies or PD-1-binding proteins or fragments thereof that are pharmaceutical equivalents or pharmaceutical alternatives whose rate and/or extent of absorption do not show a significant difference with that of cemiplimab when administered at the same molar dose under similar experimental conditions, either single dose or multiple dose. In the context of the present disclosure, the term refers to antigen-binding proteins that bind to PD-1, which do not have clinically meaningful differences with cemiplimab in their safety, purity and/or potency.

Other anti-PD-1 antibodies that can be used in the context of the methods of the present disclosure include, e.g., the antibodies referred to and known in the art as NIVOLUMAB (U.S. Pat. No. 8,008,449), PEMBROLIZUMAB (U.S. Pat. No. 8,354,509), MEDI0608 (U.S. Pat. No. 8,609,089), PIDILIZUMAB (U.S. Pat. No. 8,686,119), or any of the anti-PD-1 antibodies as set forth in U.S. Pat. Nos. 6,808,710, 7,488,802, 8,168,757, 8,354,509, 8,779,105, or 8900587.

The anti-PD-1 antibodies used in the context of the methods of the present disclosure may have pH-dependent binding characteristics. For example, an anti-PD-1 antibody for use in the methods of the present disclosure may exhibit reduced binding to PD-1 at acidic pH as compared to neutral pH. Alternatively, an anti-PD-1 antibody of the present disclosure may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to PD-1 at acidic pH as compared to neutral pH” is expressed in terms of a ratio of the K_{D} value of the antibody binding to PD-1 at acidic pH to the K_{D} value of the antibody binding to PD-1 at neutral pH (or vice versa). For example, an antibody or antigen-binding fragment thereof may be regarded as exhibiting “reduced binding to PD-1 at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained. As used herein, the expression “acidic pH” means a pH of 6.0 or less.

Bispecific Anti-CD20/Anti-CD3 Antibodies

According to certain exemplary embodiments of the present disclosure, the methods comprise administering a therapeutically effective amount of a bispecific antibody that specifically binds CD3 and CD20. Such antibodies may be referred to herein as, e.g., “anti-CD20/anti-CD3,” or “anti-CD20×CD3” or “CD20×CD3” bispecific antibodies, or other similar terminology.

As used herein, the expression “bispecific antibody” refers to an immunoglobulin protein comprising at least a first antigen-binding domain and a second antigen-binding domain. In the context of the present disclosure, the first antigen-binding domain specifically binds a first antigen (e.g., CD20), and the second antigen-binding domain specifically binds a second, distinct antigen (e.g., CD3). Each antigen-binding domain of a bispecific antibody comprises a heavy chain variable domain (HCVR) and a light chain variable domain (LCVR), each comprising three CDRs. In the context of a bispecific antibody, the CDRs of the first antigen-binding domain may be designated with the prefix “A” and the CDRs of the second antigen-binding domain may be designated with the prefix “B.” Thus, the CDRs of the first antigen-binding domain may be referred to herein as A-HCDR1, A-HCDR2, and A-HCDR3; and the CDRs of the second antigen-binding domain may be referred to herein as B-HCDR1, B-HCDR2, and B-HCDR3.

The first antigen-binding domain and the second antigen-binding domain are each connected to a separate multimerizing domain. As used herein, a “multimerizing domain” is any macromolecule, protein, polypeptide, peptide, or amino acid that has the ability to associate with a second multimerizing domain of the same or similar structure or constitution. In the context of the present disclosure, the multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group.

Bispecific antibodies of the present disclosure typically comprise two multimerizing domains, e.g., two Fc domains that are each individually part of a separate antibody heavy chain. The first and second multimerizing domains may be of the same IgG isotype, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

Any bispecific antibody format or technology may be used to make the bispecific antigen-binding molecules of the present disclosure. For example, an antibody or fragment thereof having a first antigen-binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual-acting Fab (DAF)-IgG, and Mab² bispecific formats (see, e.g., Klein et al. 2012, mAbs 4:6, 1-11, and references cited therein, for a review of the foregoing formats).

In the context of bispecific antibodies of the present disclosure, Fc domains may comprise one or more amino acid changes (e.g., insertions, deletions, or substitutions) as compared to the wild-type, naturally occurring version of the Fc domain. For example, the present disclosure includes bispecific antigen-binding molecules comprising one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications are disclosed in US Patent Publication No. 20150266966, incorporated herein in its entirety.

The present disclosure also includes bispecific antibodies comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A, and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L18M, N44S, K52N, V57M, and V821 (by IMGT; D356E, L358M, N384S, K392N, V397M, and V4221 by EU) in the case of IgG1 antibodies; N44S, K52N, and V821 (IMGT; N384S, K392N, and V4221 by EU) in the case of IgG2 antibodies; and Q15R, N44S, K52N, V57M, R69K, E79Q, and V821 (by IMGT; Q355R, N384S, K392N, V397M, R409K, E419Q, and V4221 by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H1}]-[IgG4 upper hinge]-[IgG2 lower hinge]-[IgG4 CH2]-[IgG4 CH3]. Another example of a chimeric Fc domain that can be included in any of the antigen-binding molecules set forth herein comprises, from N- to C-terminus: [IgG1 C_{H1}]-[IgG1 upper hinge]-[IgG2 lower hinge]-[IgG4 CH2]-[IgG1 CH3]. These and other examples of chimeric Fc domains that can be included in any of the antigen-binding molecules of the present disclosure are described in US Patent Publication No. 20140243504, which is herein incorporated in its entirety. Chimeric Fc domains having these general structural arrangements, and variants thereof, can have altered Fc receptor binding, which in turn affects Fc effector function.

According to certain exemplary embodiments of the present disclosure, the bispecific anti-CD20/anti-CD3 antibody, or antigen-binding fragment thereof comprises heavy chain variable regions (A-HCVR and B-HCVR), light chain variable region (LCVR), and/or complementarity determining regions (CDRs) comprising any of the amino acid sequences of the bispecific anti-CD20/anti-CD3 antibodies as set forth in US Patent Publication No. 20150266966. In certain exemplary embodiments, the bispecific anti-CD20/anti-CD3 antibody or antigen-binding fragment thereof that can be used in the context of the methods of the present disclosure comprises: (a) a first antigen-binding arm that binds to CD20 comprising the heavy chain complementarity determining regions (A-HCDR1, A-HCDR2, and A-HCDR3) of a heavy chain variable region (A-HCVR) comprising the amino acid sequence of SEQ ID NO: 11 and the light chain complementarity determining regions (LCDRs) of a light chain variable region (LCVR) comprising the amino acid sequence of SEQ ID NO: 12; and (b) a second antigen-binding arm that binds to CD3 comprising the heavy chain CDRs (B-HCDR1, B-HCDR2, and B-HCDR3) of a HCVR (B-HCVR) comprising the amino acid sequence of SEQ ID NO: 13 and the light chain CDRs of a LCVR comprising the amino acid sequence of SEQ ID NO: 12. According to certain embodiments, the A-HCDR1 comprises the amino acid sequence of SEQ ID NO: 14; the A-HCDR2 comprises the amino acid sequence of SEQ ID NO: 15; the A-HCDR3 comprises the amino acid sequence of SEQ ID NO: 16; the LCDR1 comprises the amino acid sequence of SEQ ID NO: 17; the LCDR2 comprises the amino acid sequence of SEQ ID NO: 18; the LCDR3 comprises the amino acid sequence of SEQ ID NO: 19; the B-HCDR1 comprises the amino acid sequence of SEQ ID NO: 20; the B-HCDR2 comprises the amino acid sequence of SEQ ID NO: 21; and the B-HCDR3 comprises the amino acid sequence of SEQ ID NO: 22. In yet other embodiments, the bispecific anti-CD20/anti-CD3 antibody or antigen-binding fragment thereof comprises: (a) a first antigen-binding arm comprising a HCVR (A-HCVR) comprising SEQ ID NO: 11 and a LCVR comprising SEQ ID NO: 12; and (b) a second antigen-binding arm comprising a HCVR (B-HCVR) comprising SEQ ID NO: 13 and a LCVR comprising SEQ ID NO: 12.

In one embodiment, the bispecific antibody comprises a first heavy chain comprising the HCVR of the first antigen-binding domain, a second heavy chain comprising the HCVR of the second antigen-binding domain, and a common light chain comprising the LCVR of the first and second antigen-binding domains, wherein the first heavy chain comprises the amino acid sequence of SEQ ID NO: 23. In one embodiment, the bispecific antibody comprises a first heavy chain comprising the HCVR of the first antigen-binding domain, a second heavy chain comprising the HCVR of the second antigen-binding domain, and a common light chain comprising the LCVR of the first and second antigen-binding domains, wherein the second heavy chain comprises the amino acid sequence of SEQ ID NO: 25. In one embodiment, the bispecific antibody comprises a first heavy chain comprising the HCVR of the first antigen-binding domain, a second heavy chain comprising the HCVR of the second antigen-binding domain, and a common light chain comprising the LCVR of the first and second antigen-binding domains, wherein the light chain comprises the amino acid sequence of SEQ ID NO: 24. In one embodiment, the bispecific antibody comprises a first heavy chain comprising the amino acid sequence of SEQ ID NO: 23, a second heavy chain comprising the amino acid sequence of SEQ ID NO: 25, and a common light chain comprising the amino acid sequence of SEQ ID NO: 24.

Other bispecific anti-CD20/anti-CD3 antibodies that can be used in the context of the methods of the present disclosure include, e.g., any of the antibodies as set forth in US Patent Publication Nos. 20140088295 and 20150166661. In some embodiments, the bispecific anti-CD20/anti-CD3 antibody can be REGN1979 (also known as odronextamab), as set forth in PCT publication WO2020047389A1.

Combination Therapies

The methods of the present disclosure, according to certain embodiments, comprise administering to a subject in need thereof an anti-CD20/anti-CD3 bispecific antibody or antigen-binding fragment thereof in combination with an anti-PD-1 antibody or antigen-binding fragment thereof. In some embodiments, the methods comprise administering to the subject one or more doses of an anti-CD20/anti-CD3 bispecific antibody or antigen-binding fragment thereof before administering to the subject one or more doses of an anti-PD-1 antibody or antigen-binding fragment thereof. In administering one or more doses of the anti-PD-1 antibody or antigen-binding fragment thereof, the anti-PD-1 antibody or antigen-binding fragment thereof can be administered in combination with one or more doses of the anti-CD20/anti-CD3 bispecific antibody or antigen-binding fragment thereof.

In certain embodiments, the methods of the present disclosure comprise administering the antibodies for additive or synergistic activity to treat cancer, preferably a heme cancer, more preferably, a B-cell cancer (e.g., non-Hodgkin's lymphoma or acute lymphoblastic leukemia). As used herein, the expression “in combination with” means that the anti-CD20/anti-CD3 bispecific antibody or antigen-binding fragment thereof is administered before, after, or concurrent with the anti-PD-1 antibody or antigen-binding fragment thereof. The term “in combination with” also includes sequential or concomitant administration of a bispecific anti-CD20/anti-CD3 antibody or antigen-binding fragment thereof and anti-PD-1 antibody or antigen-binding fragment thereof. For example, when administered “before” the anti-PD-1 antibody, one or more doses of the bispecific anti-CD20/anti-CD3 antibody or antigen-binding fragment thereof may be administered more than about 12 weeks, about 11 weeks, about 10 weeks, about 9 weeks, about 8 weeks, about 7 weeks, about 6 weeks, about 5 weeks, about 4 weeks, about 3 weeks, about 2 weeks, or about 1 week prior to the administration of one or more doses of the anti-PD-1 antibody or antigen-binding fragment thereof. Administration “concurrent” with the anti-PD-1 antibody means that the bispecific anti-CD20/anti-CD3 antibody or antigen-binding fragment thereof is administered to the subject in a separate dosage form within less than 5 minutes (before, after, or at the same time) of administration of the anti-PD-1 antibody or antigen-binding fragment thereof, or administered to the subject as a single combined dosage formulation comprising both the bispecific anti-CD20/anti-CD3 antibody or antigen-binding fragment thereof and the anti-PD-1 antibody or antigen-binding fragment thereof.

In certain embodiments, the methods of the present disclosure comprise administering a third therapeutic agent wherein the third therapeutic agent is an anti-cancer drug. As used herein, “anti-cancer drug” means any agent useful to treat cancer including, but not limited to, cytotoxins and agents such as antimetabolites, alkylating agents, anthracyclines, antibiotics, antimitotic agents, procarbazine, hydroxyurea, asparaginase, corticosteroids, mytotane (O,P′-(DDD)), biologics (e.g., antibodies and interferons) and radioactive agents. As used herein, “a cytotoxin or cytotoxic agent,” also refers to a chemotherapeutic agent and means any agent that is detrimental to cells. Examples include TAXOL (paclitaxel), temozolomide, cytochalasin B, gramicidin D, ethidium bromide, emetine, cisplatin, mitomycin, etoposide, teniposide, vincristine, vinblastine, colchicine, doxorubicin, daunorubicin, dihydroxy anthracene dione, mitoxantrone, mithramycin, actinomycin D, 1-dihydrotestosterone, glucocorticoids, procaine, tetracaine, lidocaine, propranolol, and puromycin and analogs or homologs thereof.

In certain embodiments, the methods of the present disclosure comprise administration of a third therapeutic agent selected from radiation, surgery, a cancer vaccine, a PD-L1 inhibitor (e.g., an anti-PD-L1 antibody), a LAG-3 inhibitor, a CTLA-4 inhibitor (e.g., ipilimumab), a TIM3 inhibitor, a BTLA inhibitor, a TIGIT inhibitor, a CD47 inhibitor, a CD38 inhibitor, a CD28 activator, an antagonist of another T-cell co-inhibitor or ligand (e.g., an antibody to CD-28, 2B4, LY108, LAIR1, ICOS, CD160 or VISTA), an indoleamine-2,3-dioxygenase (IDO) inhibitor, a vascular endothelial growth factor (VEGF) antagonist [e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab) or a small molecule kinase inhibitor of VEGF receptor (e.g., sunitinib, sorafenib, or pazopanib)], an Ang2 inhibitor (e.g., nesvacumab), a transforming growth factor beta (TGFβ) inhibitor, an epidermal growth factor receptor (EGFR) inhibitor (e.g., erlotinib, cetuximab), an agonist to a co-stimulatory receptor (e.g., an agonist to glucocorticoid-induced TNFR-related protein), an antibody to a tumor-specific antigen (e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9), a vaccine (e.g., Bacillus Calmette-Guerin, a cancer vaccine), an adjuvant to increase antigen presentation (e.g., granulocyte-macrophage colony-stimulating factor), a cytotoxin, an oncolytic virus, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine), radiotherapy, an IL-6R inhibitor (e.g., sarilumab), an IL-4R inhibitor (e.g., dupilumab), an IL-10 inhibitor, a cytokine such as IL-2, IL-7, IL-12, IL-21, and IL-15, an antibody-drug conjugate (ADC) (e.g., anti-CD19-DM4 ADC, and anti-DS6-DM4 ADC), chimeric antigen receptor T cells (e.g., CD19-targeted T cells), an anti-inflammatory drug (e.g., corticosteroids, and non-steroidal anti-inflammatory drugs), a dietary supplement such as an antioxidant, and combinations thereof.

Pharmaceutical Compositions and Administration

The present disclosure includes methods which comprise administering an anti-CD20/anti-CD3 bispecific antibody or antigen-binding fragment thereof in combination with an anti-PD-1 antibody or antigen-binding fragment thereof to a subject wherein the antibodies are contained within a separate or combined (single) pharmaceutical composition. The pharmaceutical compositions of the present disclosure may be formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN), DNA conjugates, anhydrous absorption pastes, oil-in-water, and water-in-oil emulsions, emulsions carbowax (polyethylene glycols of various molecular weights), semi-solid gels, and semi-solid mixtures containing carbowax. See also Powell et al. PDA (1998) J Pharm Sci Technol 52:238-311.

Various delivery systems are known and can be used to administer the pharmaceutical composition of the present disclosure, e.g., encapsulation in liposomes, microparticles, microcapsules, recombinant cells capable of expressing the mutant viruses, receptor-mediated endocytosis (see, e.g., Wu et al., 1987, J. Biol. Chem. 262: 4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered, and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used. In another embodiment, polymeric materials can be used; see, e.g., Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

Administration Regimens

The present disclosure includes methods comprising administering to a subject a bispecific anti-CD20/anti-CD3 antibody or antigen-binding fragment thereof at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In certain embodiments, the present disclosure includes methods comprising administering to a subject an anti-PD-1 antibody or antigen-binding fragment thereof at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In certain embodiments, the methods involve the administration an anti-CD20/anti-CD3 bispecific antibody or antigen-binding fragment thereof in combination with an anti-PD-1 antibody or antigen-binding fragment thereof at a dosing frequency of about four times a week, twice a week, once a week, once every two weeks, once every three weeks, once every four weeks, once every five weeks, once every six weeks, once every eight weeks, once every nine weeks, once every twelve weeks, or less frequently so long as a therapeutic response is achieved.

According to certain embodiments of the present disclosure, multiple doses of an anti-CD20/anti-CD3 bispecific antibody or antigen-binding fragment thereof in combination with an anti-PD-1 antibody or antigen-binding fragment thereof may be administered to a subject over a defined time course. The methods according to this aspect of the present disclosure comprise sequentially administering to a subject one or more doses of a bispecific anti-CD20/anti-CD3 antibody or antigen-binding fragment thereof in combination with one or more doses of an anti-PD-1 antibody or antigen-binding fragment thereof.

As used herein, “sequentially administering” means that each dose of the antibody is administered to the subject at a different point in time, e.g., on different days separated by a predetermined interval (e.g., hours, days, weeks or months). The present disclosure includes methods which comprise sequentially administering to the patient a single initial dose of a bispecific anti-CD20/anti-CD3 antibody or antigen-binding fragment thereof, followed by one or more secondary doses of the bispecific anti-CD20/anti-CD3 antibody or antigen-binding fragment thereof, and optionally followed by one or more tertiary doses of the bispecific anti-CD20/anti-CD3 antibody or antigen-binding fragment thereof. In certain embodiments, the methods further comprise sequentially administering to the patient a single initial dose of, followed by one or more secondary doses of an anti-PD-1 antibody or antigen-binding fragment thereof, and optionally followed by one or more tertiary doses of the anti-PD-1 antibody or antigen-binding fragment thereof.

According to certain embodiments of the present disclosure, multiple doses of a bispecific anti-CD20/anti-CD3 antibody or antigen-binding fragment thereof and an anti-PD-1 antibody or antigen-binding fragment thereof may be administered to a subject over a defined time course. The methods according to this aspect of the present disclosure comprise sequentially administering to a subject multiple doses of a bispecific anti-CD20/anti-CD3 antibody or antigen-binding fragment thereof and an anti-PD-1 antibody or antigen-binding fragment thereof. As used herein, “sequentially administering” means that each dose of the bispecific anti-CD20/anti-CD3 antibody or antigen-binding fragment thereof in combination with the anti-PD-1 antibody or antigen-binding fragment thereof is administered to the subject at a different point in time, e.g., on different days separated by a predetermined interval (e.g., hours, days, weeks or months).

The terms “initial dose,” “secondary doses,” and “tertiary doses,” refer to the temporal sequence of administration. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the antibody (bispecific antibody or anti-PD-1 antibody). In certain embodiments, however, the amount contained in the initial, secondary, and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). For example, a bispecific antibody may be administered to a patient with a B-cell cancer at a loading dose of about 1-3 mg/kg followed by one or more maintenance doses of about 0.1 to about 15 mg/kg of the patient's body weight.

In one exemplary embodiment of the present disclosure, each secondary and/or tertiary dose is administered ½ to 14 (e.g., ½, 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, a dose of a bispecific anti-CD20/anti-CD3 antibody or antigen-binding fragment thereof (and/or an anti-PD-1 antibody or antigen-binding fragment thereof) which is administered to a patient prior to the administration of the very next dose in the sequence with no intervening doses.

The methods according to this aspect of the present disclosure may comprise administering to a patient any number of secondary and/or tertiary doses of a bispecific anti-CD20/anti-CD3 antibody or antigen-binding fragment thereof (and/or an anti-PD-1 antibody or antigen-binding fragment thereof). For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In certain embodiments, one or more doses of a bispecific anti-CD20/anti-CD3 antibody or antigen-binding fragment thereof and/or an anti-PD-1 antibody or antigen-binding fragment thereof are administered at the beginning of a treatment regimen as “induction doses” on a more frequent basis (twice a week, once a week or once in 2 weeks) followed by subsequent doses (“consolidation doses” or “maintenance doses”) that are administered on a less frequent basis (e.g., once in 4-12 weeks).

The present disclosure includes methods comprising sequential administration of a bispecific anti-CD20/anti-CD3 antibody or antigen-binding fragment thereof in combination with an anti-PD-1 antibody or antigen-binding fragment thereof, to a patient to treat a B-cell cancer (e.g., non-Hodgkin's lymphoma, acute lymphoblastic leukemia). In some embodiments, the present methods comprise administering one or more doses of a bispecific anti-CD20/anti-CD3 antibody or antigen-binding fragment thereof followed by one or more doses of an anti-PD-1 antibody or antigen-binding fragment thereof. In certain embodiments, the present methods comprise administering a single dose of a bispecific anti-CD20/anti-CD3 antibody or antigen-binding fragment thereof followed by one or more doses of an anti-PD-1 antibody or antigen-binding fragment thereof. In some embodiments, one or more doses of about 0.1 mg/kg to about 15 mg/kg of the bispecific antibody may be administered followed by one or more doses of about 0.1 mg/kg to about 20 mg/kg of an anti-PD-1 antibody or antigen-binding fragment thereof to inhibit tumor growth and/or to prevent tumor recurrence in a subject with a B-cell cancer. In some embodiments, the bispecific antibody is administered at one or more doses followed by one or more doses of the anti-PD-1 antibody or antigen-binding fragment thereof resulting in increased anti-tumor efficacy (e.g., greater inhibition of tumor growth, increased prevention of tumor recurrence as compared to an untreated subject or a subject administered with either antibody as monotherapy).

Dosage

The amount of bispecific anti-CD20/anti-CD3 antibody or antigen-binding fragment thereof and/or anti-PD-1 antibody or antigen-binding fragment thereof administered to a subject according to the methods of the present disclosure is, generally, a therapeutically effective amount. As used herein, the phrase “therapeutically effective amount” means an amount of antibody (bispecific anti-CD20/anti-CD3 or antigen-binding fragment thereof or antibody anti-PD-1 antibody or antigen-binding fragment thereof) that results in one or more of: (a) a reduction in the severity or duration of a symptom of a B-cell cancer; (b) inhibition of tumor growth, or an increase in tumor necrosis, tumor shrinkage and/or tumor disappearance; (c) delay in tumor growth and development; (d) inhibit or retard or stop tumor metastasis; (e) prevention of recurrence of tumor growth; (f) increase in survival of a subject with a B-cell cancer; (g) a reduction in the use or need for conventional anti-cancer therapy (e.g., reduced or eliminated use of chemotherapeutic or cytotoxic agents); and/or (h) reduced cytokine release or reduced immune-related adverse events as compared to an untreated subject or a subject administered with either antibody as monotherapy.

In the case of a bispecific anti-CD20/anti-CD3 antibody or antigen-binding fragment thereof, a therapeutically effective amount can be from about 0.02 mg, about 0.05 mg, about 0.1 mg, about 0.5 mg, about 1 mg, about 5 mg, about 10 mg, about 20 mg, about 40 mg, about 60 mg, about 80 mg, about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 540 mg, about 560 mg, about 580 mg, about 660 mg, about 680 mg, about 700 mg, 720 mg, about 740 mg, about 760 mg, about 780 mg, about 760 mg, about 780 mg, about 800 mg, 820 mg, about 840 mg, about 860 mg, about 880 mg, about 860 mg, about 880 mg, about 900 mg, about 920 mg, about 940 mg, about 960 mg, about 980 mg, about 1000 mg, about 1020 mg, about 1040 mg, about 1060 mg, about 1080 mg, about 1100 mg, about 1120 mg, about 1140 mg, about 1160 mg, about 1180 mg, about 1200 mg of the bispecific anti-CD20/anti-CD3 antibody or antigen-binding fragment thereof.

In the case of an anti-PD-1 antibody or antigen-binding fragment thereof, a therapeutically effective amount can be from about 0.05 mg to about 1500 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 620 mg, about 640 mg, about 660 mg, about 680 mg, about 700 mg, about 720 mg, about 740 mg, about 760 mg, about 780 mg, about 800 mg, about 900 mg, about 1000 mg, about 1050 mg, about 1100 mg, or about 1200 mg of the anti-PD-1 antibody. In certain embodiments, 250 mg of an anti-PD-1 antibody is administered. In certain embodiments, 350 mg of an anti-PD-1 antibody or antigen-binding fragment thereof is administered.

The amount of either bispecific anti-CD20/anti-CD3 antibody or antigen-binding fragment thereof or anti-PD-1 antibody or antigen-binding fragment thereof contained within the individual doses may be expressed in terms of milligrams of antibody per kilogram of subject body weight (i.e., mg/kg or mpk). In certain embodiments, either anti-PD-1 antibody or bispecific anti-CD20/anti-CD3 antibody used in the methods of the present disclosure may be administered to a subject at a dose of about 0.0001 to about 100 mg/kg of subject body weight. For example, anti-PD-1 antibody or antigen-binding fragment thereof may be administered at a dose of about 0.1 mg/kg to about 20 mg/kg of a patient's body weight. The bispecific anti-CD20/anti-CD3 antibody or antigen-binding fragment thereof may be administered at a dose of about 0.1 mg/kg to about 15 mg/kg of a Patient's body weight.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

As used herein, the term “agent” denotes a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

As used herein, the terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

As used herein, the term “disease” is intended to be generally synonymous and is used interchangeably with, the terms “disorder” and “condition” (as in medical condition), in that all reflect an abnormal condition (e.g., inflammatory disorder) of the human or animal body or of one of its parts that impairs normal functioning, is typically manifested by distinguishing signs and symptoms, and causes the human or animal to have a reduced duration or quality of life.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

As used herein, the term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present disclosure within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of one or more components of the present disclosure, and are physiologically acceptable to the subject. Supplementary active compounds may also be incorporated into the compositions.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

As used herein, the singular forms “a,” “an,” and “the” include plural references unless the context clearly dictates otherwise.

As used herein, the terms “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like are used repeatedly. Such phrases do not necessarily refer to the same embodiment, but they may unless the context dictates otherwise.

As used herein, the terms “and/or” or “/” means any one of the items, any combination of the items, or all of the items with which this term is associated.

As used herein, the word “substantially” does not exclude “completely,” e.g., a composition which is “substantially free” from Y may be completely free from Y. Where necessary, the word “substantially” may be omitted from the definition of the present disclosure.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

The use of any and all examples, or exemplary language (e.g., “such as”) provided herein, is intended merely to better illuminate the present disclosure and does not pose a limitation on the scope of the present disclosure unless otherwise claimed. No language in the specification should be construed as indicating any non-claimed element as essential to the practice of the present disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present disclosure is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

The following examples are put forth so as to provide those of ordinary skill in the art with a complete disclosure and description of how to make and use the methods and compositions of the present disclosure and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

Targeted therapies for cancer hold the potential for specifically identifying and destroying cancer cells while minimizing adverse side effects. However, these novel therapies present unique challenges for determining a starting dose that is safe for patients and avoids unwanted immune cell activation. This disclosure demonstrates how these challenges were addressed using human immune cell-based assays to assess predicted levels of immune cell activation across a range of drug concentrations and to support the minimum anticipated biological effect level (MABEL) approach as well as aid in clinical study design. Though the aim of combinatorial treatment approaches is to enhance immune cell activity against cancer cells, potential risks associated with excessive immune cell activation need to be assessed. The assay disclosed herein was designed to address safety concerns and provide a rationale for a proposed dosing scheme involving cemiplimab+odronextamab.

Current immunotherapeutic molecules for cancer treatment are aimed at enhancing T-cell mediated tumor clearance through one or more of the following approaches: (1) TCR/CD3: T-cells are activated through T-cell receptor-complex recognition of MHC peptide-complex (called ‘signal 1’) or bypassed through the activation of associated CD3 signaling molecules by using a bispecific antibody targeting tumor-associated antigen (TAA) and CD3; (2) Co-stimulatory signals: Engagement of co-stimulatory receptors such as CD28 serve as ‘signal 2’ in T-cell activation which is required for an enhanced T-cell mediated immune response. CD28 can be activated through natural ligands CD80 & CD86 expressed on antigen-presenting cells (APCs), or bypassed through a bispecific antibody targeting tumor-associated antigen (TAA) and CD28; and (3) Inhibitory signals: Inhibitory receptors, whose expression is significantly enhanced on activated T-cells, such as PD-1, CTLA-4 (competes with CD28 for CD80 & CD86 binding), and LAG-3 reduce T-cell activation when engaged with their corresponding ligands and therefore need to be blocked to release the inhibition (brake) of T-cell activation.

The potential for odronextamab to be more efficacious in combination with PD-1 blockade was investigated, especially if the tumor expresses multiple TAAs and/or is PD-L1+. To that end, primary human T-cell assays were developed to address these concerns and assist in the development of a dosing strategy.

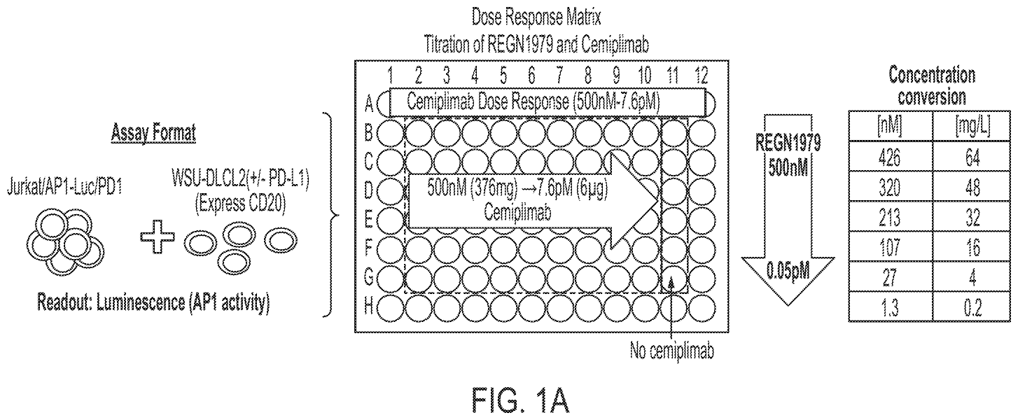

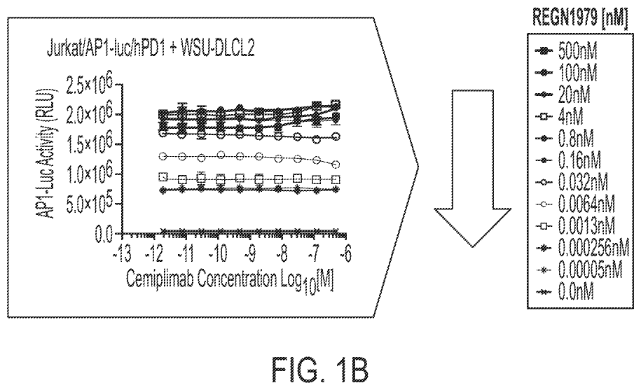

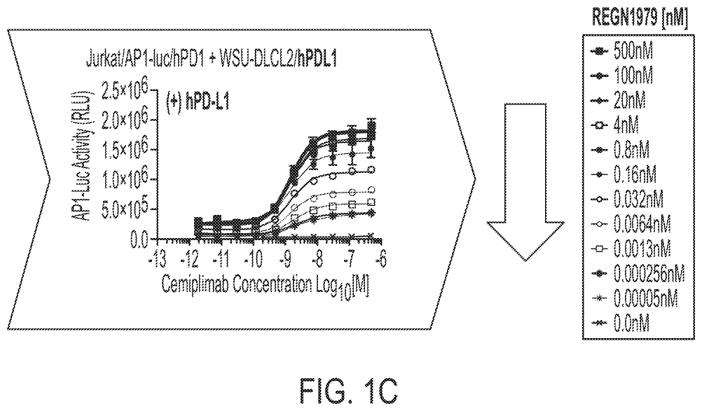

FIGS. 1A, 1B, and 1C show an engineered reporter assay to evaluate odronextamab (REGN1979)+cemiplimab. Engineered reporter T-cells (Jurkat/AP1-Luc/PD-1) were incubated with WSU-DLCL2 cells or WSU-DLCL2/PD-L1 cells, and a titration of odronextamab and cemiplimab, with cemiplimab concentrations plotted on the x-axis and the different colored lines representing the odronextamab titration. FIG. 1A shows titrations of odronextamab ranging from 500 nM to 0.05 pM. FIG. 1B shows odronextamab stimulates AP1-Luc activity from T-cells incubated with WSU-DLCL2 cells, with little to no impact being observed from the titration of cemiplimab, which was expected due to lack of PD-L1 in the system. FIG. 1C shows that odronextamab stimulation of AP1-Luc in T-cells was significantly decreased in the presence of PD-L1 on WSU-DLCL2, which facilitated PD-1 activation and repression of T-cell/AP1-Luc activity. The activity was restored with cemiplimab.

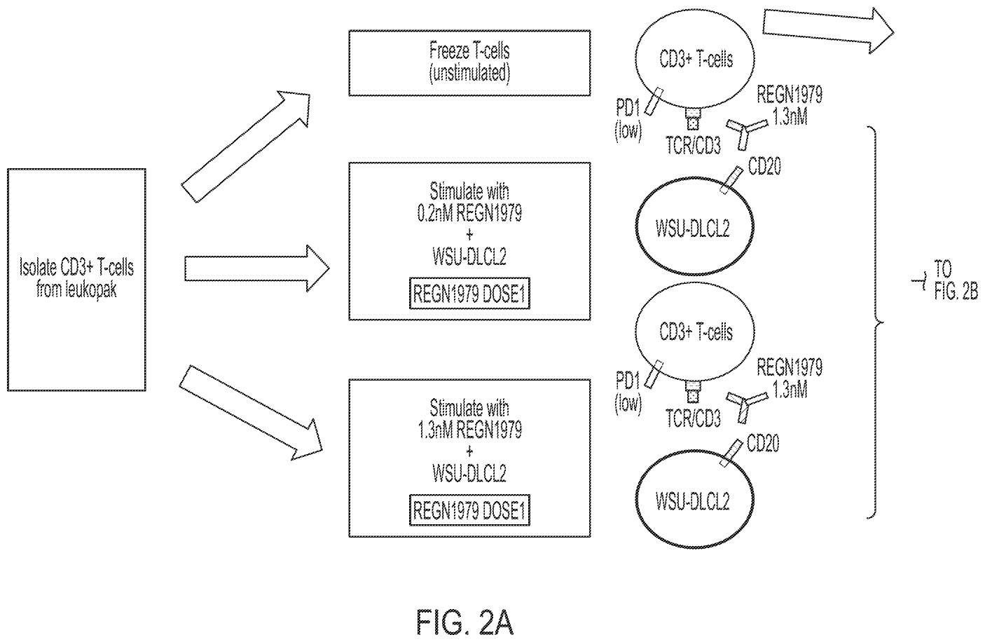

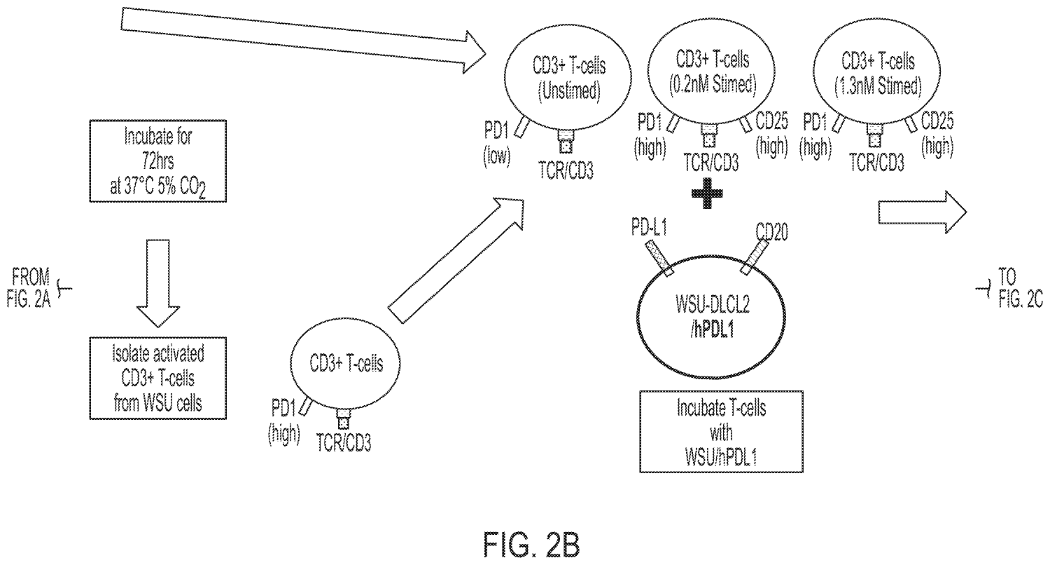

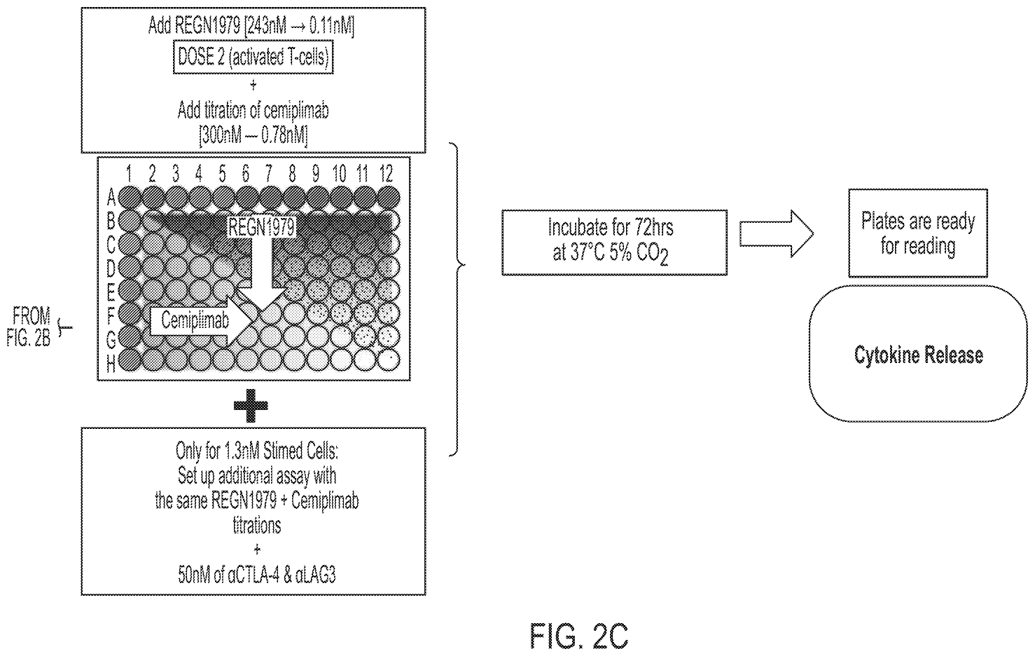

FIGS. 2A, 2B, and 2C show primary CD3+ T-cell assay to evaluate odronextamab (REGN1979)+cemiplimab. CD3+ T-cells were isolated from healthy donor PBMC's and frozen down (assay condition 1) or incubated with WSU-DLCL2 cells in the presence of 200 pM (condition 2) or 1.3 nM (condition 3) odronextamab for 72 hours at 37° C. and under 5% CO2. T-cells are then re-isolated from WSU-DLCL2 (note molecules of odronextamab still on-board T-cells) and used in a bioassay in the presence of either WSU-DLCL2 or WSUDLCL2/PD-L1. Unstimulated and pre-stimulated T-cells were also stained at this point for CD25, CTLA-4, PD-1, and LAG3. Odronextamab titration range from 243 nM to 0.11 nM. For T-cells obtained from condition 1, this is their first dose of odronextamab. For T-cells obtained from condition 2 & 3, this was their second dose of odronextamab. Cemiplimab titration ranges from 300 nM-0.78 pM. All T-cells only receive one dose of cemiplimab. Additionally, T-cells obtained from condition 3 were also subjected to +/−anti-CTLA-4 & anti-LAG3 combination (50 nM each). Assay plates were incubated for 72 hours at 37° C. and under 5% CO2. The supernatant was collected from all plates, and an ALPHALISA was performed to detect IL-2 and IFNγ release. Only assay data from WSU/PD-L1 conditions are shown. WSU-DLCL2 wildtype data did not show any response to cemiplimab due to the assay being a PD-L1 negative. The result of 0.2 nM pre-stimulation is similar to that of the 1.3 nM condition.

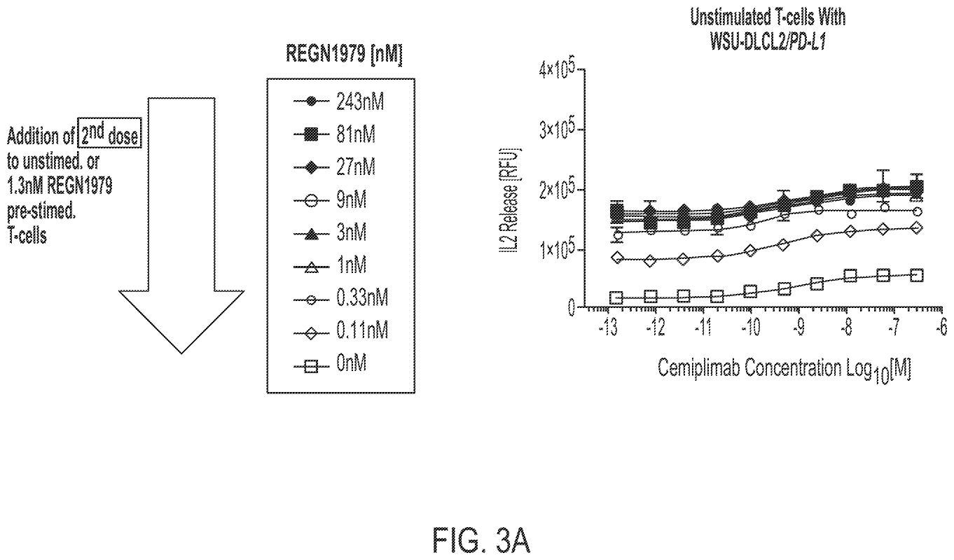

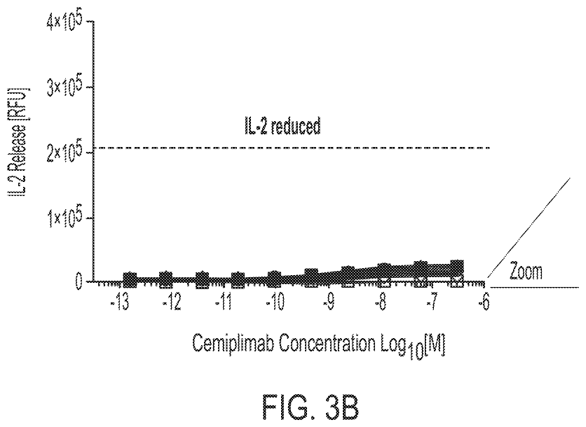

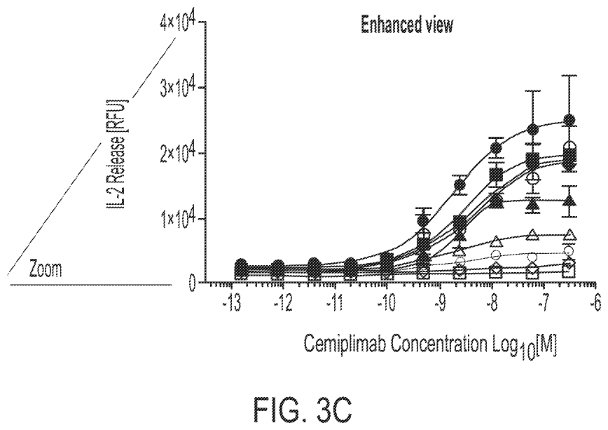

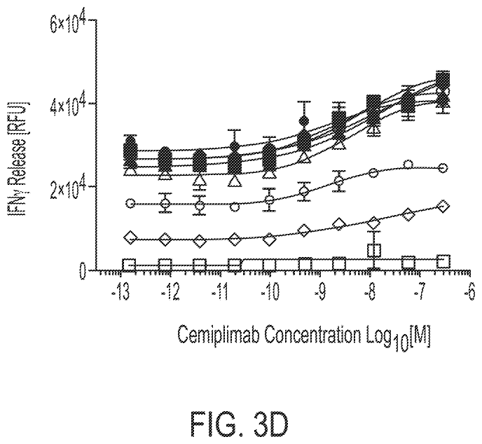

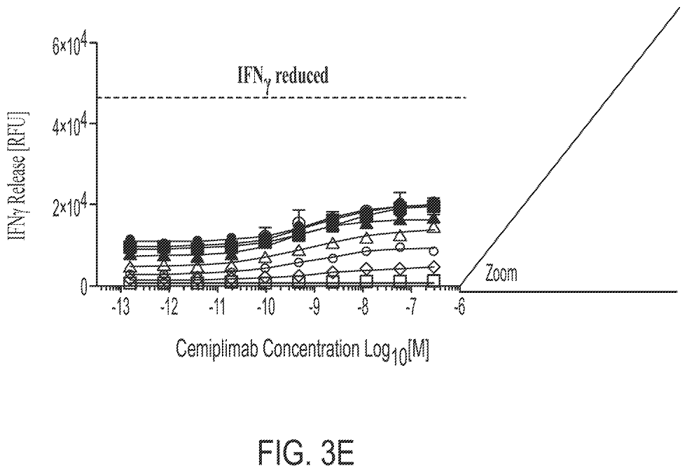

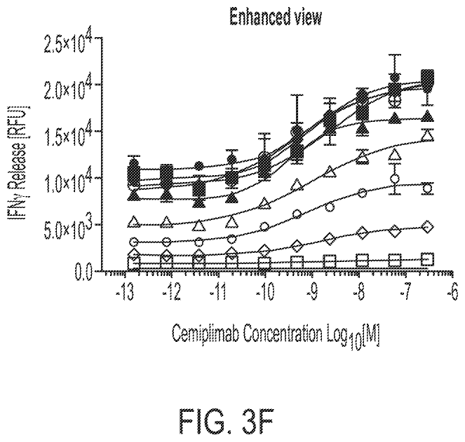

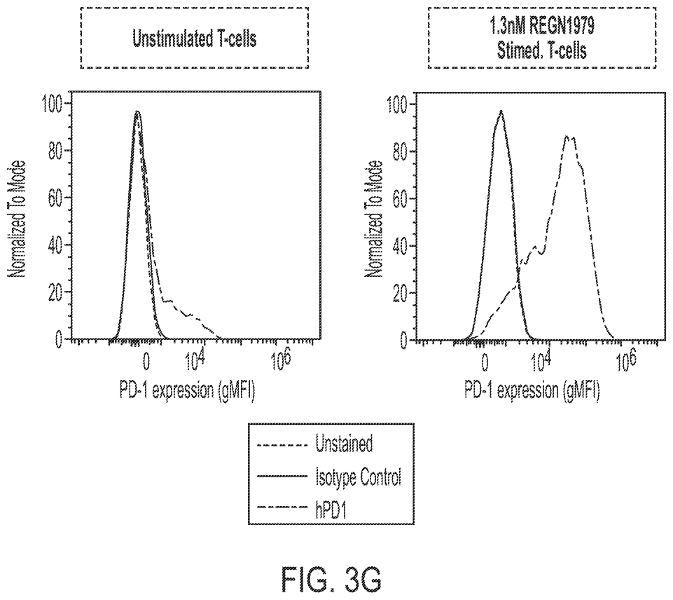

FIGS. 3A, 3B, 3C, 3D, 3E, 3F, and 3G show the results from odronextamab (REGN1979)+cemiplimab lead-in dosing assay. T-cells exposed to lead-in dose of odronextamab experienced cytokine rescue through cemiplimab treatment. T-cells which were isolated, frozen down, and only received one dose of odronextamab, minimally respond to cemiplimab treatment in the presence of WSU/PD-L1 cells (FIG. 3A). FIG. 3B shows IL-2 levels in T-cells pre-stimulated with an initial dose of 1.3 nM odronextamab without combination. There is a response in the absence of cemiplimab, with the 1.3 nM pre-treated T-cells that is detectable, with increasing concentrations of odronextamab. The enhanced view of the 1.3 nM prestimulated T-cells reveals high sensitivity to cemiplimab treatment in combination with odronextamab (FIG. 3C). IFNγ release followed a similar trend as IL-2 with cytokine levels dropping significantly with pretreatment and becoming more sensitive to cemiplimab combination (FIGS. 3D, 3E, and 3F). Odronextamab monotherapy in pre-stimulated cells led to increases in IFNγ release with increasing odronextamab doses. Staining unstimulated and pre-stimulated T-cells revealed higher surface expression of PD1 on pre-stimulated T-cells (FIG. 3G).

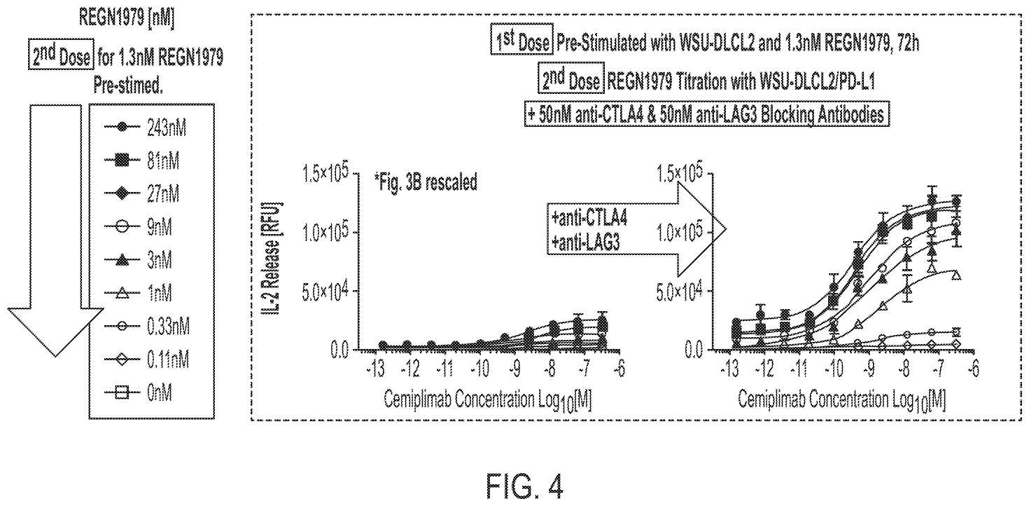

FIG. 4 shows cytokine response from pre-stimulation could be further enhanced by blocking CTLA-4 and LAG-3. 1.3 nM pre-stimulated T-cells were treated with a titration of odronextamab (REGN1979) and cemiplimab (FIG. 3B). IL-2 response decreased dramatically compared to unstimulated T-cells however were more sensitive to cemiplimab (FIG. 3A). By adding 50 nM of anti-CTLA-4 and ant-LAG3 antibody with the titration of odronextamab and cemiplimab, IL-2 release was dramatically increased. IFNγ release also experiences similar rescue through treatment. Based on staining data, the expression of PD-1 (FIG. 3G), CTLA-4, and LAG-3, all increase when T-cells were stimulated with odronextamab+WSU cells compared to unstimulated cells.

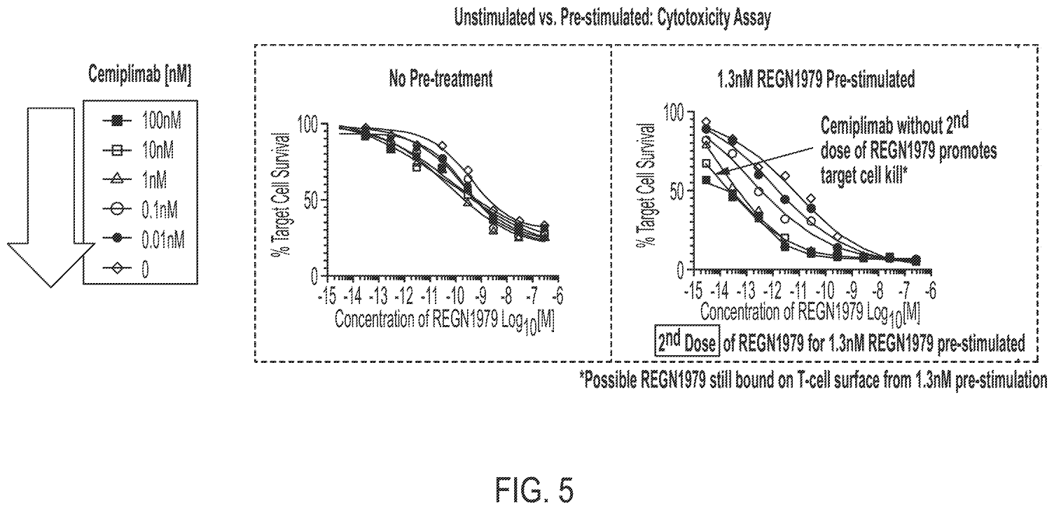

FIG. 5 shows that T-cell cytotoxicity was not compromised by pre-treatment of odronextamab (REGN1979) and was enhanced by cemiplimab. Untreated and preactivated human T-cells and Vybrant CFDA-SE labeled WSU/PD-L1 cells were plated at the E:T ratio of 5:1 with a 10-fold serial dilution of odronextamab and cemiplimab (starting at 300 nM and 100 nM, respectively) for 3 days at 37° C. 5% CO2 in complete media. At the end of the culture, surviving WSU/PDL1 cells and T cell activation and proliferation were assessed using flow cytometry. Unstimulated T-cells kill target cells with odronextamab treatment in a dose-dependent manner. cemiplimab treatment did not impact killing. Pre-stimulated T-cells were sensitive to cemiplimab treatment at all doses tested and enhanced target cell kill in combination with odronextamab.

Through the bioassays as described above, T-cell mediated cytokine responses from the tested molecules as both monotherapies and combination therapies could be detected. For the odronextamab+cemiplimab program, data could be rapidly generated through the Jurkat/AP1-luc reporter assay, which provided in vitro evidence for the rationale behind this combination. Based on the generated data, these molecules were tested using primary human T-cells following a ‘lead-in’ dosing model, which generated dose titration curves for cemiplimab in the presence of odronextamab in vitro. This bioassay led to key findings that (i) follow-up doses of odronextamab produced a significantly weaker IL-2 response; (ii) odronextamab-treated T cells are increasingly susceptible to cemiplimab treatment; and (iii) T cells significantly upregulate inhibitory checkpoint receptors when stimulated with odronextamab.

### Example 2

A Quantitative Systems Pharmacology (QSP) Modeling Framework for Evaluation of Cytokine Release Mediated by Odronextamab (REGN1979)/Cemiplimab (REGN2810) Combination Therapies in Patients with B-NHL

QSP is an emerging mathematical modeling approach which integrates the current understanding of disease biology, drug mechanism of action, and in vivo/in vitro/clinical data in order to generate hypotheses and predictions that address specific questions in both preclinical research and clinical development. This example describes a study using a QSP modeling approach to evaluate clinical cytokine profiles following odronextamab monotherapy and combination therapy with odronextamab and cemiplimab, a PD-1 inhibitor.

Objectives: Odronextamab (REGN1979) is a fully human IgG4-based bispecific antibody that binds to CD3⁺ T-cells and CD20⁺ B-cells, targeting CD20⁺ tumor cells via T-cell-mediated cytotoxicity. The engagement of CD3 on T-cells and CD20 on B-cells activates T-cells. During T-cell activation, inflammatory cytokines are secreted, which can result in a significant but temporary increase in the circulating cytokine concentrations and may lead to a systemic inflammatory response, known as CRS. This study used a QSP modeling approach to evaluate cytokine profiles, as represented by IL-6, following odronextamab monotherapy and combination therapy with odronextamab and cemiplimab (REGN2810; a PD-1 inhibitor) in patients with B-NHL. An objective of this work is to predict cytokine profiles with different dosing schedules.

Methods: A QSP model was developed that integrates pharmacokinetics of odronextamab and cemiplimab, dynamics of T-cell and B-cell, disease characteristics of NHL, and cytokine data from odronextamab monotherapy study (NCT02290951) and odronextamab/cemiplimab combination study (NCT02651662). In the QSP model, the mechanism of cytokine release was described as (1) the formation of trimolecular synapse when odronextamab binds to both CD3 on T-cells and target CD20 on B-cells and (2) regulation of T-cell activation by the PD-1 pathway. To describe cytokine profiles under the treatment of odronextamab/cemiplimab combination, it was hypothesized that cemiplimab increased the stimulation of IL-6 production by activating more T-cells and reduced the inhibitory effect of the immune system to attenuate the cytokine release. Concentrations of IL-6, odronextamab, and cemiplimab from NHL patients under monotherapy and in combination, and in vivo in vitro data (e.g., tumor growth rate, CD20/CD3 expression levels, T/B-cell baseline levels, and drug affinity data) were used to calibrate the model.

The QSP model integrated data from several sources, including data describing odronextamab and cemiplimab mechanisms of action and pharmacokinetics, T cell and B-cell dynamics, CD20/CD3/PD-1 expression levels, disease characteristics of NHL, and clinical cytokine data from Phase 1 studies of odronextamab monotherapy (NCT02990951) and odronextamab/cemiplimab combination therapy (NCT02651662). Data sources used to inform this QSP model are shown in Table 1.

TABLE 1
Data sources for the QSP model

| Property | Data source |
|---|---|
| Odronextamab PK | FIH odronextamab monotherapy study |
| | (NCT02990951) |
| Cemiplimab PK | Cemiplimab population-PK analysis report |
| Odronextamab cytokine | FIH odronextamab monotherapy study |
| data | (NCT02990951) |
| Odronextamab/cemiplimab | Odronextamab/cemiplimab combination |
| cytokine data | study (NCT02651662) |
| Odronextamab dosing | FIH odronextamab monotherapy study |
| regimen | (NCT02990951) |
| Cemiplimab dosing | Odronextamab/cemiplimab combination |
| regimen | study (NCT02651662) |
| Baseline B-cells | FIH odronextamab monotherapy study |
| | (NCT02990951) |
| Baseline T cells | FIH odronextamab monotherapy study |
| | (NCT02990951) |
| Baseline SPDs | FIH odronextamab monotherapy study |
| | (NCT02990951) |
| Odronextamab affinity | Odronextamab in vitro study |
| Cemiplimab affinity | Cemiplimab in vitro study |
| PD-1/PDL-1 affinity | Literature data (Cheng et al., J Biol Chem, |
| | 288(17): 11771-78 (2013)) |
| CD20/CD30 density | Literature data (Betts et al., AAPS J, 12: 66 |
| | (2019); Huh et al., Am J Clin Pathol, 116(3): |
| | 437-43 (2001)) |
| PD-1 density | Literature data (Cheng et al., J Biol Chem, |
| | 288(17): 11771-78 (2013)) |

FIH, first-in human; PK, pharmacokinetics; SPD, sum of products of diameters.

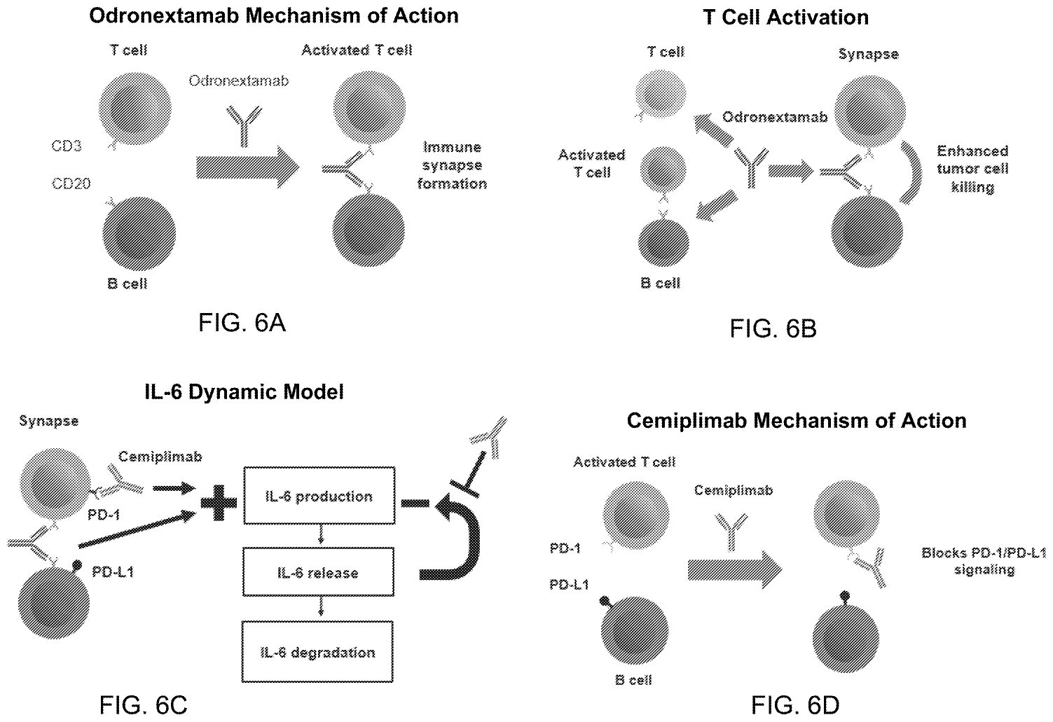

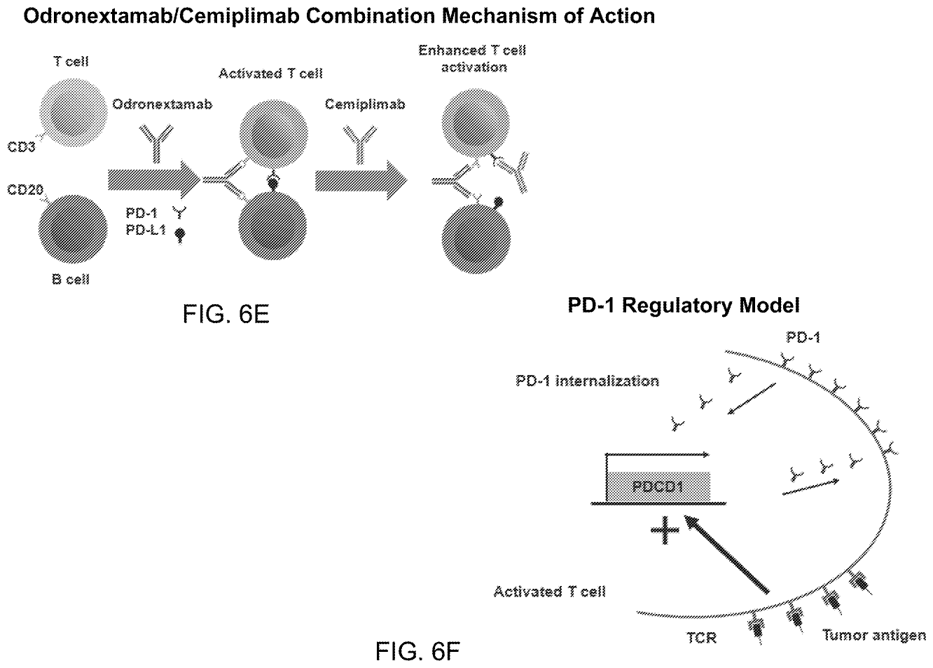

Conceptually, the QSP model was built using different modules to capture the interactions between each drug alone, and in combination, with malignant lymphocytes. The mechanism of cytokine release was described with two components: (i) cytokine release due to the formation of a trimolecular synapse when odronextamab binds to both CD3 on T cells and CD20 on B-cells following monotherapy (FIGS. 6A-6B); and (ii) cytokine profiles following treatment with odronextamab/cemiplimab in combination; cemiplimab affects the regulation of T cell activation via interaction with the PD-1 pathway (FIGS. 6B-6F).

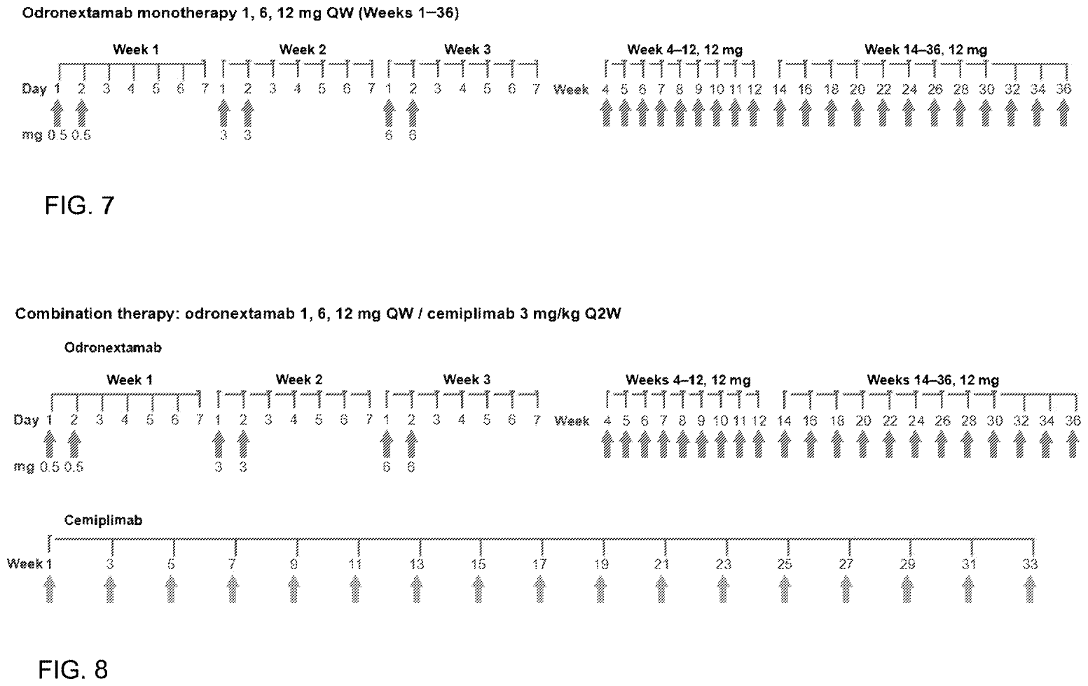

Multiple cytokines are released under odronextamab dosing (e.g., IL-6, IL-10, IFN-γ, TNF-α). To simplify the model, IL-6 was selected as a representative cytokine, because most cytokine time profiles are similar, and IL-6 is regarded as a key cytokine associated with CRS (Cheng et al., J Biol Chem, 288(17):11771-78 (2013)). The model was calibrated using IL-6 concentration data from patients with B-NHL treated in Phase 1 studies of odronextamab monotherapy and odronextamab/cemiplimab combination therapy. Representative regimens are described in FIG. 7 and FIG. 8. Other in vivo/in vitro data included tumor growth rate, CD20/CD3 expression levels, T cell and B-cell baseline levels, and drug affinity analyses. (Cheng et al., J Biol Chem, 288(17):11771-78 (2013)).

In the simulation, 300 virtual patients with aggressive NHL were created using a Monte Carlo sampling approach.

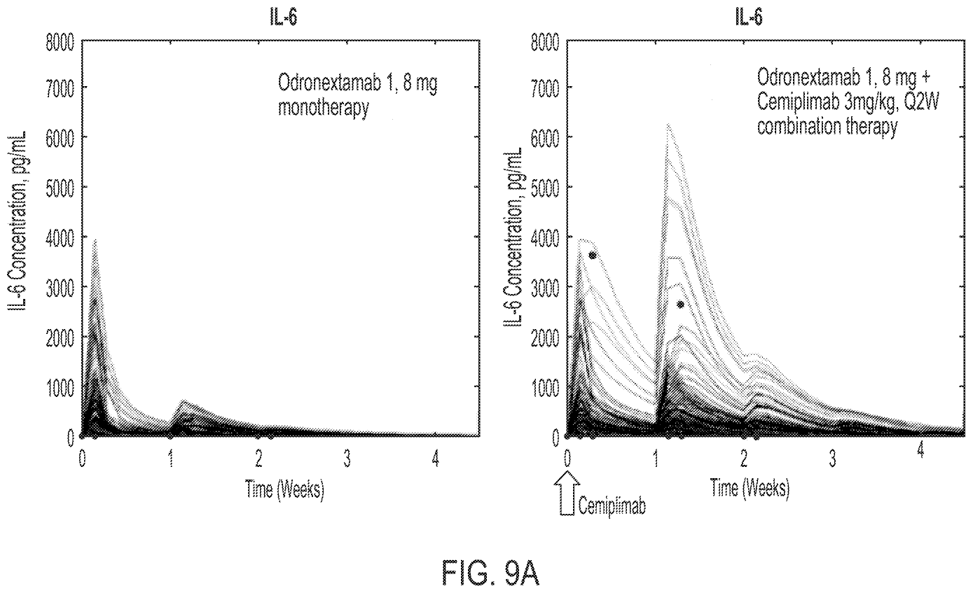

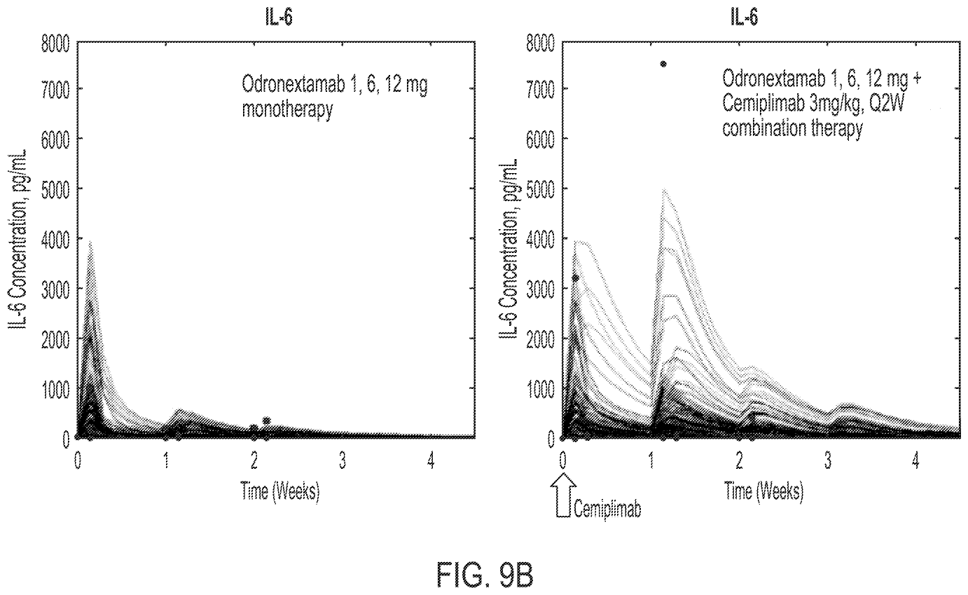

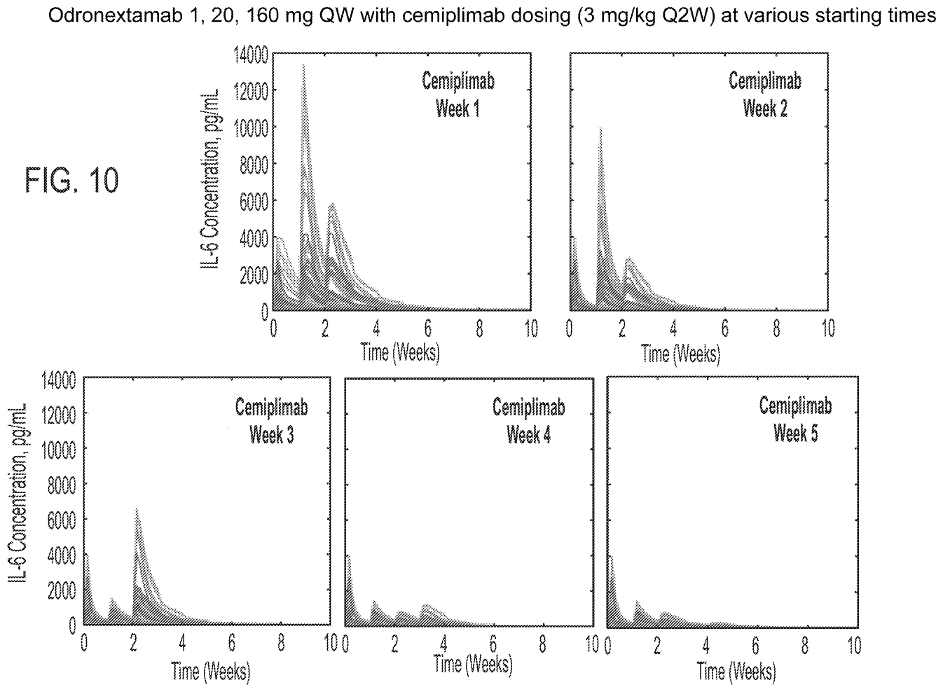

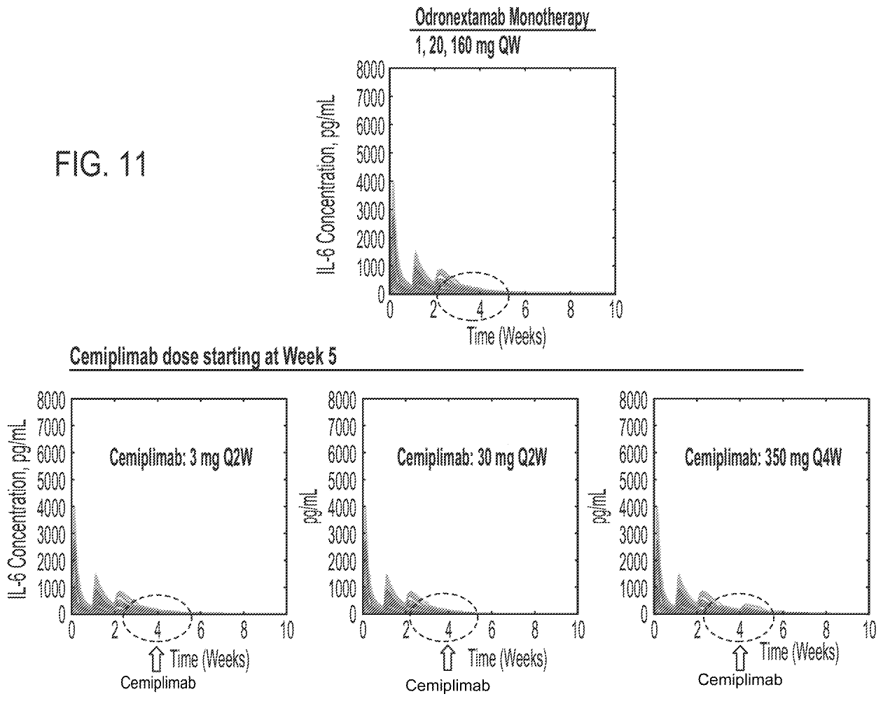

Results: In a simulated population of 300 patients with aggressive NHL, the model predicted that: (i) Week 1, Day 1 IL-6 concentrations were higher when odronextamab and cemiplimab were dosed in combination compared with odronextamab monotherapy, consistent with observed data from Phase 1 trials (FIGS. 9A-9B); (ii) IL-6 release would be attenuated by delayed introduction of cemiplimab co-administration, with progressive decreases in IL-6 release with increasing delay in cemiplimab initiation (FIG. 10); and (iii) when starting cemiplimab administration from Week 5 (after odronextamab monotherapy lead-in), the impact of cemiplimab dose level (3, 30, and 350 mg) on IL-6 concentration is expected to be minimal (FIG. 11).

The model-based simulations indicate that under odronextamab monotherapy, IL-6 concentrations mostly peaked on day 1 following an initial split dose of 1 mg in week 1. The cytokine concentrations reduced over time, even with increased odronextamab doses up to 10- and 100-fold in week 2 and week 3, respectively. When odronextamab and cemiplimab were dosed simultaneously on day 1 week 1, the mean peaks of IL-6 concentrations were higher than that of odronextamab monotherapy due to cemiplimab effect on PD-1/PD-L1 signaling pathway, consistent with observed data.

Conclusions: This exploratory model-based assessment provides insights for IL-6 dynamics following treatment with odronextamab and cemiplimab. The model-based simulations predict a higher risk of IL6 release if administration of odronextamab and cemiplimab occur simultaneously from Week 1, Day 1. Inclusion of an odronextamab lead-in phase before addition of cemiplimab can limit cytokine concentrations to levels similar to that of odronextamab monotherapy.

### REFERENCES

1. Smith E J, et al. Sci Rep. 2015; 5:17943.
2. Choi B D, et al. Expert Opin Biol Ther. 2011; 11(7):843-53.
3. Shimabukuro-Vornhagen A, et al. J Immunother Cancer. 2018; 6(1):56.
4. Cheng X, et al. J Biol Chem. 2013; 288(17):11771-78.
5. Betts A, et al. AAPS J. 2019; 21:66.
6. Huh Y O, et al. Am J Clin Pathol. 2001; 116(3):437-43.

We claim:

1. A method of treating or inhibiting the growth of a tumor, comprising:
(a) selecting a subject with cancer;
(b) administering to the subject a therapeutically effective amount of a bispecific anti-CD20/anti-CD3 antibody or antigen-binding fragment thereof comprising a first antigen-binding arm that specifically binds CD20 and a second antigen-binding arm that specifically binds CD3; and
(c) after step (b), administering to the subject an antibody or antigen-binding fragment thereof that specifically binds programmed death 1 (PD-1);
wherein the method treats or inhibits the growth of a tumor and ameliorates cytokine release syndrome (CRS) in the subject.

2. A method of ameliorating cytokine release syndrome (CRS) in a subject with a tumor, comprising:
(a) selecting a subject with cancer;
(b) administering to the subject a therapeutically effective amount of a bispecific anti-CD20/anti-CD3 antibody or antigen-binding fragment thereof comprising a first antigen-binding arm that specifically binds CD20 and a second antigen-binding arm that specifically binds CD3; and
(c) after step (b), administering to the subject an antibody or antigen-binding fragment thereof that specifically binds programmed death 1 (PD-1).

3. The method of claim 1 or 2, wherein step (c) further comprises administering to the subject the anti-PD-1 antibody or antigen-binding fragment thereof in combination with the bispecific antibody or antigen-binding fragment thereof.

4. The method of any one of claims 1-3, wherein the bispecific antibody or antigen-binding fragment thereof is administered to the subject at least about 1 week prior to administering the anti-PD-1 antibody or antigen-binding fragment thereof.

5. The method of any one of claims 1-4, wherein each of the bispecific antibody or antigen-binding fragment thereof and the anti-PD-1 antibody or antigen-binding fragment thereof is administered in one or more doses to the subject.

6. The method of claim 5, wherein the first dose of the bispecific antibody or antigen-binding fragment thereof is administered to the subject about 1 week, about 2 weeks, about 3 weeks, about 4 weeks, about 5 weeks, about 6 weeks, about 7 weeks, or about 8 weeks prior to administering the first dose of the anti-PD-1 antibody or antigen-binding fragment thereof.

7. The method of claim 5, wherein the first dose of the bispecific antibody or antigen-binding fragment thereof is administered to the subject about 5 weeks prior to administering the first dose of the anti-PD-1 antibody or antigen-binding fragment thereof.

8. The method of any one of claims 1-7, wherein the bispecific antibody or antigen-binding fragment thereof is administered to the subject in one or more doses of about 0.1 mg/kg to about 15 mg/kg of body weight of the subject.

9. The method of any one of claims 1-7, wherein the bispecific antibody or antigen-binding fragment thereof is administered to the subject in one or more doses of about 1 mg to about 800 mg.

10. The method of claim 8 or 9, wherein the bispecific antibody or antigen-binding fragment thereof is administered to the subject once a day, once every two days, once every three days, once every five days, once every week, once every two weeks, once every three weeks, or once every four weeks.

11. The method of claim 8 or 9, wherein each dose of the one or more doses of the bispecific antibody or antigen-binding fragment thereof is administered 0.5 to 12 weeks after the immediately preceding dose.

12. The method of any one of claims 8-11, wherein at least one of the one or more doses of the bispecific antibody or antigen-binding fragment thereof comprises a dose having a greater amount of the bispecific antibody or antigen-binding fragment thereof than the immediately preceding dose thereof.

13. The method of any one of claims 8-12, wherein at least one of the one or more doses of the bispecific antibody or antigen-binding fragment thereof is administered in two or more split doses.

14. The method of claim 13, wherein at least one of the two or more split doses comprises the identical amount of the bispecific antibody or antigen-binding fragment thereof.

15. The method of claim 13, wherein at least one of the two or more split doses is administered at least about 0.5 days after the immediately preceding dose.

16. The method of claim 15, wherein at least one of the two or more split doses is administered about 1 day after the immediately preceding dose.

17. The method of any one of claims 1-16, wherein the anti-PD-1 antibody or antigen-binding fragment thereof is administered to the subject in one or more doses of about 0.1 mg/kg to about 20 mg/kg of body weight of the subject.

18. The method of any one of claims 1-16, wherein the anti-PD-1 antibody or antigen-binding fragment thereof is administered to the subject in one or more doses of about 1 mg to about 1500 mg.

19. The method of claim 17 or 18, wherein the anti-PD-1 antibody or antigen-binding fragment thereof is administered to the subject once a day, once every two days, once every three days, once every five days, once every week, once every two weeks, once every three weeks, once every four weeks, once every five weeks or once every six weeks.

20. The method of claim 17 or 18, wherein at least one of the one or more doses of the anti-PD-1 antibody or antigen-binding fragment thereof is administered 0.5 to 12 weeks after the immediately preceding dose.

21. The method of any one of claims 17-20, wherein at least one of the one or more doses of the anti-PD-1 antibody or antigen-binding fragment thereof comprises a dose having a greater amount of the anti-PD-1 antibody or antigen-binding fragment thereof than the immediately preceding dose thereof.

22. The method of any one of claims 1-21, wherein the bispecific antibody or antigen-binding fragment thereof or the anti-PD-1 antibody or antigen-binding fragment thereof are administered to the subject intravenously, subcutaneously, or intraperitoneally.

23. The method of claim 1, wherein the subject has cytokine release syndrome.

24. The method of any one of claims 1-23, wherein the tumor comprises a B-cell cancer.

25. The method of claim 24, wherein the B-cell cancer is selected from Hodgkin's lymphoma, non-Hodgkin's lymphoma, follicular lymphoma, small lymphocytic lymphoma, lymphoplasmacytoid lymphoma, marginal zone lymphoma, mantle cell lymphoma, diffuse large B-cell lymphoma, B-cell lymphomas, lymphomatoid granulomatosis, Burkitt's lymphoma, acute lymphoblastic leukemia, hairy cell leukemia, and B-cell chronic lymphocytic leukemia.

26. The method of any one of claims 1-25, wherein the subject is resistant to, inadequately responsive to, or relapsed after prior therapy.

27. The method of any one of claims 1-26, wherein the subject has been treated with prior anti-CD20 therapy.

28. The method of claim 27, wherein the anti-CD20 therapy comprises an anti-CD20 antibody.

29. The method of any one of claims 1-28, wherein the treatment produces a therapeutic effect selected from delay in tumor growth, reduction in tumor cell number, tumor regression, increase in survival, partial response, and complete response.

30. The method of any one of claims 1-29, wherein the treatment leads to an effect selected from reduced cytokine release, reduced release of IL-2, IL-6, IL-10, TNF-α and/or IFN-γ, reduced administration of dexamethasone, corticosteroids or an analgesic, reduced number of immune related adverse events, and reduced number of ≥Grade 3 adverse events.

31. The method of claim 29 or 30, wherein tumor growth is delayed by at least 10 days as compared to an untreated subject.

32. The method of any one of claims 1-31, wherein the tumor growth is inhibited by at least 50% as compared to an untreated subject.

33. The method of claim 32, wherein the tumor growth is inhibited by at least 50% as compared to a subject administered with either the bispecific antibody or antigen-binding fragment thereof or the anti-PD-1 antibody or antigen-binding fragment thereof as monotherapy.

34. The method of any one of claims 1-33, further comprising administering to the subject a third therapeutic agent or therapy.

35. The method of claim 34, wherein the third therapeutic agent or therapy is selected from radiation, surgery, a chemotherapeutic agent, a cancer vaccine, a PD-L1 inhibitor, a LAG-3 inhibitor, a CTLA-4 inhibitor, a TIM3 inhibitor, a BTLA inhibitor, a TIGIT inhibitor, a CD47 inhibitor, a CD28 activator, a CD38 inhibitor, a GITR agonist, an indoleamine-2,3-dioxygenase (IDO) inhibitor, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an antibody to a tumor-specific antigen, Bacillus Calmette-Guerin vaccine, granulocyte-macrophage colony-stimulating factor, a cytotoxin, an interleukin 6 receptor (IL-6R) inhibitor, an interleukin 4 receptor (IL-4R) inhibitor, an IL-10 inhibitor, IL-2, IL-7, IL-12, IL-21, IL-15, an antibody-drug conjugate, an oncolytic virus, an anti-inflammatory drug, a dietary supplement, and combinations thereof.

36. The method of any one of claims 1-35, wherein the first antigen-binding arm of the bispecific antibody or antigen-binding fragment thereof comprises three heavy chain CDRs (A-HCDR1, A-HCDR2, and A-HCDR3) and three light chain CDRs (LCDR1, LCDR2, and LCDR3), and wherein A-HCDR1 comprises the amino acid sequence of SEQ ID NO: 14; A-HCDR2 comprises the amino acid sequence of SEQ ID NO: 15; A-HCDR3 comprises the amino acid sequence of SEQ ID NO: 16; LCDR1 comprises the amino acid sequence of SEQ ID NO: 17; LCDR2 comprises the amino acid sequence of SEQ ID NO: 18; and LCDR3 comprises the amino acid sequence of SEQ ID NO: 19.

37. The method of any one of claims 1-36, wherein the first antigen-binding arm of the bispecific antibody or antigen-binding fragment thereof comprises a heavy chain variable region (A-HCVR) having the amino acid sequence of SEQ ID NO: 11 and a light chain variable region (LCVR) having the amino acid sequence of SEQ ID NO: 12.

38. The method of any one of claims 1-37, wherein the second antigen-binding arm of the bispecific antibody or antigen-binding fragment thereof comprises three heavy chain CDRs (B-HCDR1, B-HCDR2, and B-HCDR3) and three light chain CDRs (LCDR1, LCDR2, and LCDR3), and wherein B-HCDR1 comprises the amino acid sequence of SEQ ID NO: 20; B-HCDR2 comprises the amino acid sequence of SEQ ID NO: 21; B-HCDR3 comprises the amino acid sequence of SEQ ID NO: 22; LCDR1 comprises the amino acid sequence of SEQ ID NO: 17; LCDR2 comprises the amino acid sequence of SEQ ID NO: 18; and LCDR3 comprises the amino acid sequence of SEQ ID NO: 19.

39. The method of any one of claims 1-38, wherein the second antigen-binding arm of the bispecific antibody or antigen-binding fragment thereof comprises a heavy chain variable region (B-HCVR) having the amino acid sequence of SEQ ID NO: 13 and a light chain variable region (LCVR) having the amino acid sequence of SEQ ID NO: 12.

40. The method of any one of claims 1-39, wherein the bispecific antibody comprises a first heavy chain comprising the HCVR of the first antigen-binding domain, a second heavy chain comprising the HCVR of the second antigen-binding domain, and a common light chain comprising the LCVR of the first and second antigen-binding domains, wherein the first heavy chain comprises the amino acid sequence of SEQ ID NO: 23.

41. The method of any one of claims 1-40, wherein the bispecific antibody comprises a first heavy chain comprising the HCVR of the first antigen-binding domain, a second heavy chain comprising the HCVR of the second antigen-binding domain, and a common light chain comprising the LCVR of the first and second antigen-binding domains, wherein the second heavy chain comprises the amino acid sequence of SEQ ID NO: 25.

42. The method of any one of claims 1-41, wherein the bispecific antibody comprises a first heavy chain comprising the HCVR of the first antigen-binding domain, a second heavy chain comprising the HCVR of the second antigen-binding domain, and a common light chain comprising the LCVR of the first and second antigen-binding domains, wherein the light chain comprises the amino acid sequence of SEQ ID NO: 24.

43. The method of any one of claims 1-35, wherein the bispecific antibody is odronextamab.

44. The method of any one of claims 1-43, wherein the anti-PD-1 antibody or antigen-binding fragment thereof comprises three heavy chain complementarity determining regions (HCDR1, HCDR2, and HCDR3) and three light chain complementarity determining regions (LCDR1, LCDR2, and LCDR3), and wherein HCDR1 comprises the amino acid sequence of SEQ ID NO: 3; HCDR2 comprises the amino acid sequence of SEQ ID NO: 4; HCDR3 comprises the amino acid sequence of SEQ ID NO: 5; LCDR1 comprises the amino acid sequence of SEQ ID NO: 6; LCDR2 comprises the amino acid sequence of SEQ ID NO: 7; and LCDR3 comprises the amino acid sequence of SEQ ID NO: 8.

45. The method of any one of claims 1-44, wherein the anti-PD-1 antibody or antigen-binding fragment thereof comprises a heavy chain variable region (HCVR) having the amino acid sequence of SEQ ID NO: 1 and a light chain variable region (LCVR) having the amino acid sequence of SEQ ID NO: 2.

46. The method of any one of claims 1-43, wherein the anti-PD-1 antibody comprises a heavy chain having the amino acid sequence of SEQ ID NO: 9 and a light chain having the amino acid sequence of SEQ ID NO: 10.

47. The method of any one of claims 1-43, wherein the anti-PD-1 antibody is cemiplimab.
